(19) United States
(12) Patent Application Publication
(10) Pub. No.: US 2018/0070830 A1
(43) Pub. Date: Mar. 15, 2018

(54) SYSTEMS AND METHODS FOR TIME-RESOLVED DIFFUSE CORRELATION SPECTROSCOPY

(71) Applicant: The General Hospital Corporation, Boston, MA (US)

(72) Inventors: Jason Sutin, Cambridge, MA (US); Maria Angela Franceschini, Winchester, MA (US); David Boas, Winchester, MA (US); Juliette Selb, Charlestown, MA (US)

(21) Appl. No.: 15/564,489

(22) PCT Filed: Apr. 11, 2016
(86) PCT No.: PCT/US16/26933

§ 371 Date: Oct. 5, 2017

Related U.S. Application Data
(60) Provisional application No. 62/145,104, filed on Apr. 9, 2015.

(51) Int. Cl. A61B 5/00; A61B 5/026; A61B 5/1455; A61B 5/021

(52) U.S. Cl. CPC A61B 5/0075; A61B 5/0261; A61B 5/021; A61B 5/14553; A61B 5/4064; A61B 5/72

(57) ABSTRACT

The present disclosure generally relates to improvements to systems and methods for measuring the dynamic properties of scattering particles within a medium, including fluid flow. Specifically, the present disclosure relates to systems and methods for time-resolved diffuse correlation spectroscopy. This disclosure provides systems and methods for determining dynamics in a target medium. The systems and methods can utilize time-resolved diffuse correlation spectroscopy.

### CROSS-REFERENCE TO RELATED APPLICATIONS

This application is related to, claims priority to, and incorporates herein by reference for all purposes U.S. Provisional Patent Application No. 62/145,104, filed Apr. 9, 2015.

### STATEMENT REGARDING FEDERALLY SPONSORED RESEARCH

This invention was made with government support under P41-EB015896, R01-HD042908, and R01-EB001954 awarded by the National Institutes of Health. The government has certain rights in the invention.

### BACKGROUND

The present disclosure generally relates to improvements to systems and methods for measuring the dynamic properties of scattering particles within a medium, including fluid flow. Specifically, the present disclosure relates to systems and methods for time-resolved diffuse correlation spectroscopy.

Physiological monitoring of the delivery and consumption of oxygen by organs has great significance for many applications, including, but not limited to, healthcare, rehabilitation, performance monitoring, and athletic training. For example, cerebral monitoring will significantly improve the management of patients with brain injury, patients at risk for brain injury, and patients undergoing routine general anesthesia and surgical procedures that alter cerebral oxygen delivery. Near-infrared spectroscopy (NIRS) has been used for more than two decades to monitor tissue oxygenation (SO₂) as a surrogate of blood flow (BF) and oxygen delivery. While NIRS oximeters show significant correlation between SO₂ and arterial blood pressure, oxygenation is not the same as blood flow or metabolism. The SO₂—BF relationship is affected by changes in oxygen consumption (i.e. the metabolic rate of oxygen, MRO₂), arterial oxygenation (SaO₂), hemoglobin in the blood (HGB), and in the relative volumes of the arterial and venous compartments. It is therefore highly desirable to measure blood flow in tissues, either alone, or in combination with a measurement of oxygenation. Furthermore, it is desirable for these measurements to be made in a continual or continuous manner to enable applications such as monitoring during intensive care or in the field. It is also highly desirable for these measurements to be made non-invasively, for example with an instrument probe external to the body for measuring blood flow or blood flow and oxygenation of an internal organ with minimal influence from overlying layers of skin, muscle, and/or bone, or minimally invasive, for example with a laparoscope or endoscope.

Multiple methods for quantitatively measuring blood flow are well-known, but most are either invasive and/or non-continuous. Modern techniques for measuring cerebral blood flow in humans include radiographic clearance methods, magnetic resonance imaging (MRI) spin-labeling, transcranial Doppler ultrasound (TCD), thermal diffusion, and laser Doppler flowmetry (LDF). Radiographic clearance methods are the oldest techniques and generally involve measuring the rate of washout of a radioisotope tracer. Radiographic methods have the advantage of quantitatively measuring absolute regional blood flow throughout the entire brain, including deep brain structures. However, they have the disadvantages of requiring radiation, being expensive and slow, and cannot be performed continuously or at the bed-side or in the field. MRI arterial spin-labeling (ASL) is another non-invasive method to measure regional blood flow throughout the entire brain. However, the accuracy and precision of the method is poor, quantitation is difficult, and the dynamic range of measurable flow rates are limited by the lifetime of the spin label. As with radiographic methods, ASL cannot be deployed at the bedside or in the field. Transcranial Doppler ultrasound measures cerebral blood flow velocity in large cerebral arteries as a surrogate for global cerebral blood flow. While TCD is non-invasive, it cannot provide regional measures of microcirculation and is confounded by changes in vessel caliber. TCD also requires significant expertise for proper use, and is difficult to apply continuously for extended periods of time as the ultrasonic probe must be maintained in a proper orientation with the insonated cerebral artery. TCD also has difficulty measuring flow velocity in the anterior cerebral arterial which supplies blood to the clinically important frontal region of the brain. Finally, due to normal anatomical variations, skull thickness in about 15% of subjects is too thick to allow blood flow measurements by TCD.

The most clinically used invasive measures of blood flow are thermal diffusion and laser Doppler flowmetry. Thermal diffusion measures absolute blood flow in a small region localized around a probe. To measure cerebral blood flow, the thermal diffusion probe must be inserted a few centimeters into the brain. LDF is similarly invasive, requiring a hole burred through the skull and a probe placed directly on the surface of the brain itself. Since the LDF detection volume is small (˜1 mm³), LDF flow values are highly variable, with values dependent on slight differences in the local vascular anatomy underneath the probe and not necessarily representative of the microcirculation of the tissue of interest. LDF has the further disadvantage of not being calibrated to absolute flow. While thermal diffusion and LDF can provide continuous measurements, the invasiveness of these techniques clearly limit their application to severely ill patients.

Recently a new technology has been developed for measuring blood flow non-invasively by using ultrasound to perturb NIRS light as it travels through tissue. However, the physical principle of this approach is fundamentally different than the approach of the present disclosure.

Optical methods are well-known for measuring fluid flow, particularly laser Doppler flowmetry and diffuse correlation spectroscopy (DCS). However, both methods depend on either a priori knowledge of the optical properties (optical absorption and scattering coefficients, etc. . . . ) of the subject or specimen in which flow is measured or actual measurement of the optical properties of the subject by independent means. This is disadvantageous for several reasons. If the optical properties of the subject are simply assumed or taken from an average of measurements from multiple or representative samples, then the discrepancy between the actual optical properties of the subject and the values assumed in the analysis lead to greater inaccuracies in the determination of flow. The resulting inter-subject variability make comparison of flow between different subjects more difficult. The actual optical properties of the subjects can be measured by other means, but this increases the cost and complexity of flow determination. Furthermore, if the optical properties of the specimen vary with time and if the optical properties of the subject are not measured simultaneously or nearly-simultaneously with flow, then the analysis of flow will be inaccurate and intra-subject variability will increase. Thus, it is highly desirable to measure the actual optical properties of the subject simultaneously or nearly-simultaneously with flow.

Typically in laser Doppler flowmetry, a long coherence length source of light illuminates the specimen and backscattered light is measured from a location in the immediate vicinity of the location where the illumination is directed onto the sample. For example, a common LDF configuration uses a multimodal optical fiber to deliver light to the subject and a second multi-mode fiber, laterally displaced about 0.25 mm from the source fiber, to receive light transmitted from the source through the tissue. Other configurations use free space or single-mode optical fibers or a combination of fiber optics and free-space. Regardless of the means of delivering and detecting light, the close proximity of the light source and detectors has the advantage of increasing the flux of light at the detector, since the intensity of the scattered light decreases roughly exponentially with distance from the illumination source. Thus, in LFD a relatively large amount of light is detected and analog detection schemes are typically employed. Light scattering from particles moving in the specimen introduces a flow-dependent Doppler broadening to the scattered light, the amount of which can be determined by a variety of means. In principle, the optical spectra of the scattered light could be measured directly. In practice, more commonly, fluctuations in the detected intensity are measured and then temporal power spectrum or auto correlation can be computed to quantify the dynamic scattering. Typically, LDF is realized in the single or few scattering regime and often simple moment analysis is used to quantify flow.

Diffuse correlation spectroscopy is an optical flow measurement technique related to LDF, with the principal difference that DCS is realized in the multiply scattering regime to enable measurement of deep tissue. In DCS, source-detector separations are typically up to a hundred-fold greater than the separations used in LDF. The depth of sensitivity of the measurement into the tissue is roughly approximately half the source detector separation distance, so 3 cm separations are typically adequate for a non-invasive transcranial measurement of cerebral blood flow in adults. Thus, DCS is an improvement over LDF because DCS enables non-invasive measurement of cerebral perfusion. However, as with NIRS, this improvement comes with the disadvantage that a majority of the measured DCS signal arises from intervening superficial layers of tissue and not from the tissue of interest. For cerebral measurements, the influence of the scalp on DCS signals is less than that on NIRS signals due to the vastly different physiological flow rates. Despite the positive impact of the flow differential on DCS signals, in the prior art, the majority of the transcranial DCS signal in an adult originates from the scalp and not the brain. One aspect of this invention specifically improves the sensitivity of flow measurements to the tissue of interest.

Another advantage of DCS is that its larger sensitive volume provides greater spatial averaging over the tissue region of interest, leading to improved robustness of the flow measurement with respect to LDF. A disadvantage of DCS is that the larger separations lead to greater light loss through tissue and the small light coherence areas require small aperture detectors for adequate contrast of the DCS signal. The net result is a relatively low detected photon flux, requiring more expensive detectors and typically photon counting. As a result, lower signals are obtained and more source power and/or averaging (either in time and/or multiple detectors) is required to achieve equivalent signal-to-noise ratios.

Since DCS is performed in the multiply scattering regime, quantification of flow is typically performed by determining a blood flow index (BFi) from the intensity temporal correlation function of the detected intensities. It is highly desirable to absolutely quantify BFi so the values can be accurately compared from subject to subject, for example such that normative blood flow levels can be defined and thresholds established for clinical intervention. To make an accurate determination or estimation of flow, the scattering coefficient of the examined tissue must be determined or assumed. It is well known that errors in flow estimates are proportional to errors in the scattering coefficient. Furthermore, errors in flow estimates are also proportional to errors in the absorption coefficient.

Time-resolved NIRS (TR-NIRS or time-resolved spectroscopy, TRS) are a family of techniques to measure the optical properties of turbid media and tissues. TR-NIRS techniques are further subdivided into those based on time-domain (TD) and those based on frequency-domain (FD). TR-NIRS techniques have the common requirement of a pulsed light source with a pulse width faster than the time of flight of the photons through the media to be examined or a light modulated is modulated with sufficient frequency for an appreciable phase shift to occur during passage through the media. Generally in TD-NIRS, the time of flight of photons through the tissue is measured and the resulting histogram, referred to variously as the temporal point spread function (TPSF), is analyzed for the absorption (μₐ) and/or scattering (μₛ′) coefficients, or equivalents. Generally in FD-NIRS, various combinations of AC intensity, DC intensity, and phase shift are measured and analyzed for μₐ and/or μₛ′, or equivalents. It is well known that the TD and FD families can overlap in either measurement techniques, analysis techniques, or both, for example when the harmonic content of a pulsed laser source is used for FD measurements or when the TPSF is Fourier transformed and analyzed in the frequency domain. Both TD and FD techniques are well known to be performed in either the analog or digital measurement and/or analysis domain or any combination therein.

Continuous wave NIRS (CW-NIRS) is a family of techniques where changes in optical absorbance are measured using a continuous or quasi-continuous light source. For the purposes of CW techniques, a quasi-continuous light source is one that has nearly constant intensity or is modulated or pulsed with a period of modulation or pulse width slower than the time of flight of the photons through the media to be examined. TR-NIRS has the desirable property of measuring the optical scattering in tissue, while CW-NIRS must use assumptions or the results from independent measurement of scattering by another method.

By using multiple measurement wavelengths, NIRS, whether CW or TR or any combination therein, the concentrations of oxyhemoglobin and deoxyhemoglobin can be measured and/or estimated, along with SO₂, oxygen extraction fraction (OEF), etc. By combining these measurements or estimates with measurements or estimates of blood flow, for example from DCS, the metabolic rate of oxygen (MRO₂) can be determined. Determination of MRO₂ is very desirable because MRO₂ represents the actual metabolism of the tissue or organ and is representative of its actual performance, physiological or pathological state, whereas other measure, such as oxygen saturation of hemoglobin are convolved with the mechanics of oxygen delivery as well as consumption. When measured in the brain, MRO₂ is known as the cerebral metabolic rate of oxygen (CMRO₂).

In the prior art, since the inception of the use of DCS and LDF to measure fluid flow, the light source for DCS or LDF has been assumed to require a coherence length much longer than the path length distribution width of the light through the tissue. Therefore, prior to this invention, the leading minds in the field thought that it was not feasible to use pulsed or FD-modulated illumination for DCS.

There exists a need for new and improved systems and methods for measurement of fluid flow, and specifically, non-invasive measurement of blood flow.

### SUMMARY

The present invention overcomes drawbacks of previous technologies by providing systems and methods for time-resolved diffuse correlation spectroscopy (TR-DCS).

In one aspect, the present disclosure provides a TR-DCS system. The system can include one or more of the following: a TR-DCS source, the TR-DCS source configured to transmit pulses of light into a target medium, the pulses of light having a pulse length of between 1 ps and 10 ns; a TR-DCS detector, the TR-DCS detector configured to receive the pulses of light from the target medium and to generate a TR-DCS detector signal in response to receiving the pulses of light; a memory storing one or more equations relating time of flight and correlation to dynamics of scattering particles within the target medium; and a processor coupled to the TR-DCS detector and the memory, the processor configured to determine a dynamics of the target medium using the TR-DCS detector signal and the one or more equations.

In another aspect, the present disclosure provides a TR-DSC source. The TR-DCS source can include a light source configured to transmit pulses of light having a pulse length of between 1 ps and 10 ns into a target medium; and a trigger source configured to generate a trigger signal that triggers the light source to emit the pulses of light and/or is correlated to the emission of the pulses of light from the light source. The light source can be further configured to transmit the pulses of light into the target medium with either an average power of between 10 μW and 10 W or a coherence length of between 0.01 mm and a transform limit of the pulses of light.

In a further aspect, the present disclosure provides a method for making a TR-DCS measurement of scattering particle dynamics within a target medium. The method can include one or more of the following steps: a) coupling a TR-DCS source and a TR-DCS detector to the target medium, the TR-DCS source configured to emit pulses of light having a pulse length of between 1 ps and 10 ns; b) transmitting a first pulse of light from the TR-DCS source into the target medium, the first pulse of light comprising a plurality of photons; c) receiving at least a portion of the plurality of photons at the TR-DCS detector after passing through the target medium, thereby generating a TR-DCS detector signal including a timing information and a correlation information for the at least a portion of the plurality of photons; d) determining, using a processor, the timing information, the correlation information, and one or more equations relating time of flight and correlation to dynamics, a dynamics of the target medium; and e) generating a report including the dynamics of the target medium.

In yet another aspect, the present disclosure provides a method of making a TR-DCS measurement of a target medium. The method can include one or more of the following steps: a) coupling a TR-DCS source to the target medium; b) emitting a first pulse of light from the TR-DCS source into the target medium, the first pulse of light having a first pulse length of between 1 ps and 10 ns, the first pulse of light comprising a plurality of photons; c) multiplexing at least a portion of the plurality of photons after passing through the target medium with a reference pulse of light emitted from the TR-DCS source or a different light source, thereby generating a multiplexed optical signal, the reference pulse of light has not passed through the target medium, the reference pulse of light having a reference pulse length that is the same or different than the first pulse length, the reference pulse length is between 1 ps and 100 ns; d) receiving the multiplexed optical signal at an optical detector, thereby generating a detector signal including timing information and correlation information for the at least a portion of the plurality of photons; e) determining, using a processor, the timing information, the correlation information, and one or more equations relating time of flight and correlation to dynamics, dynamics of the target medium; and f) generating a report including the dynamics of the target medium.

In yet a further aspect, the present disclosure provides a method of making a time-gated or time-tagged DCS measurement of a target medium. The method can include one or more of the following steps: a) coupling a DCS source and a DCS detector to a surface of the target medium; b) transmitting a plurality of photons from the DCS source into the target medium, each emitted photon emitted at a known emission time; c) waiting a length of time for at least a portion of the plurality of photons to propagate through the medium from the DCS source to the DCS detector; d) detecting the at least a portion of the plurality of photons using the DCS detector, each detected photon of the at least a portion of the plurality of photons detected at a known detection time; e) determining a transit time for each of the at least a portion of the plurality of photons; f) determining, using photons where the transit time that exceeds a pre-determined threshold, an inner dynamics of an inner portion of the target medium relative to the surface, or, using photons where the transit time is less than a pre-determined threshold, a superficial dynamics of a superficial layer of the target medium relative to the surface; and g) generating a report including the inner dynamics or the superficial dynamics.

The foregoing and other advantages of the disclosure will appear from the following description. In the description, reference is made to the accompanying drawings which form a part hereof, and in which there is shown by way of illustration a preferred embodiment of the disclosure. Such embodiment does not necessarily represent the full scope of the disclosure, however, and reference is made therefore to the claims and herein for interpreting the scope of the disclosure.

### BRIEF DESCRIPTION OF THE DRAWINGS

The present disclosure will hereafter be described with reference to the accompanying drawings, wherein like reference numerals denote like elements.

FIG. 1 is a schematic of a system, in accordance with the present disclosure.

FIG. 2 is a schematic of a system, in accordance with the present disclosure.

FIG. 3 is a schematic representation of various emission profiles, in accordance with the present disclosure.

FIG. 4 is a schematic of a system having multiple wavelengths and wavelength-specific filters, in accordance with the present disclosure.

FIG. 5 is a flowchart illustrating a method, in accordance with the present disclosure.

FIG. 6 is a flowchart illustrating a method, in accordance with the present disclosure.

FIG. 7 is a flowchart illustrating a method, in accordance with the present disclosure.

FIG. 8 is a flowchart illustrating a method, in accordance with the present disclosure.

FIG. 9 is a plot comparing signals at various separation distances, the plot showing signal versus time of flight, as described in Example 1.

FIG. 10 is a plot comparing signals at various separation distances, the plot showing cumulative signal versus time of flight, as described in Example 1.

FIG. 11 is a bar graph comparing the sensitivity of the TR-DCS method to brain blood flow in comparison with CW NIRS and CW DCS, as described in Example 1.

FIG. 12 is a plot of a TPSF, as described in Example 2.

FIG. 13 is a plot of an autocorrelation function, as described in Example 2.

FIG. 14 is a plot of a TPSF showing a time gate, as described in Example 2.

FIG. 15 is a plot of an autocorrelation function for varying gate widths, as described in Example 2.

FIG. 16 is a plot of a TPSF showing various time gates having the same width but different relative starting times, as described in Example 2.

FIG. 17 is a plot of the autocorrelation functions for the time gates shown in FIG. 16, as described in Example 2.

FIG. 18 is a plot of the amplitude of the correlation functions for the time gates shown in FIG. 16, as described in Example 2.

FIG. 19 is a plot of the path-length-dependent autocorrelation functions for the time gates shown in FIG. 16, as described in Example 2.

FIG. 20 is a plot of slopes from the fits shown in FIG. 19, as described in Example 2.

FIG. 21 is a plot of the TPSF described in Example 3.

FIG. 22 is a plot of autocorrelation functions described in Example 3.

FIG. 23 is a plot of the TPSF described in Example 4.

FIG. 24 is a plot of the amplitudes of the autocorrelation functions described in Example 4.

FIG. 25 is a plot of the slope of g₁ₛ versus the path length, as described in Example 4.

FIG. 26 is a plot illustrating the sensitivity of the methods to hypercapnia in rats, as described in Example 5.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive, so recitation of a value of between 1 and 10 includes the values 1 and 10. Disclosure of multiple alternative ranges having different maximum and/or minimum values contemplates all combinations of the maximum and minimum values disclosed therein. For example, recitation of a value of between 1 and 10 or between 2 and 9 contemplates a value of between 1 and 9 or between 2 and 10 in addition to the positively recited values, unless explicitly stated to the contrary.

This disclosure provides systems and methods for time-resolved diffuse correlation spectroscopy (TR-DCS) or time-resolved laser Doppler flowmetry (TR-LDF). For the sake of brevity, the disclosure is described in terms of TR-DCS, but the features of the present disclosure are also applicable to TR-LDF. In areas where TR-DCS is mentioned, TR-LDF is also expressly contemplated. For example, the TR-DCS source and TR-DCS detector are also contemplated as a TR-LDF source and TR-LDF detector, respectively. A person having ordinary skill in the art will appreciate the differences between DCS and LDF are applicable to the present disclosure, and can propagate those differences throughout the present disclosure. Non-limiting examples of some typical differences between DCS and LDF can include, but are not limited to, the following: LDF can typically use multimode optical fibers as waveguides, whereas DCS can typically use single-mode fibers; LDF can typically use analog detection, whereas DCS can typically use photon counting detection; and LDF can often be performed in the low-scattering regime, whereas DCS can often be performed in the multiply-scattering regime. Again, for clarity, the present disclosure is intended to encompass TR-DCS and TR-LDF systems and methods, and wherever TR-DCS (or TD-DCS or other reference to DCS) is recited, TR-LDF (or TD-LDF or corresponding other reference to LDF) is expressly contemplated. Should a person having ordinary skill in the art recognize that substituting LDF for DCS requires modification of the system or method in a way known to those having ordinary skill in the art, then that modification is also expressly contemplated.

As used herein, the terms “time of flight” and “pathlength” are used interchangeably to refer to the length of time and/or the distance that a photon travels from the source to detector.

As used herein, the terms “timing” and “phase shift” are used interchangeably to refer to the relative timing of coherent light sources.

### Systems

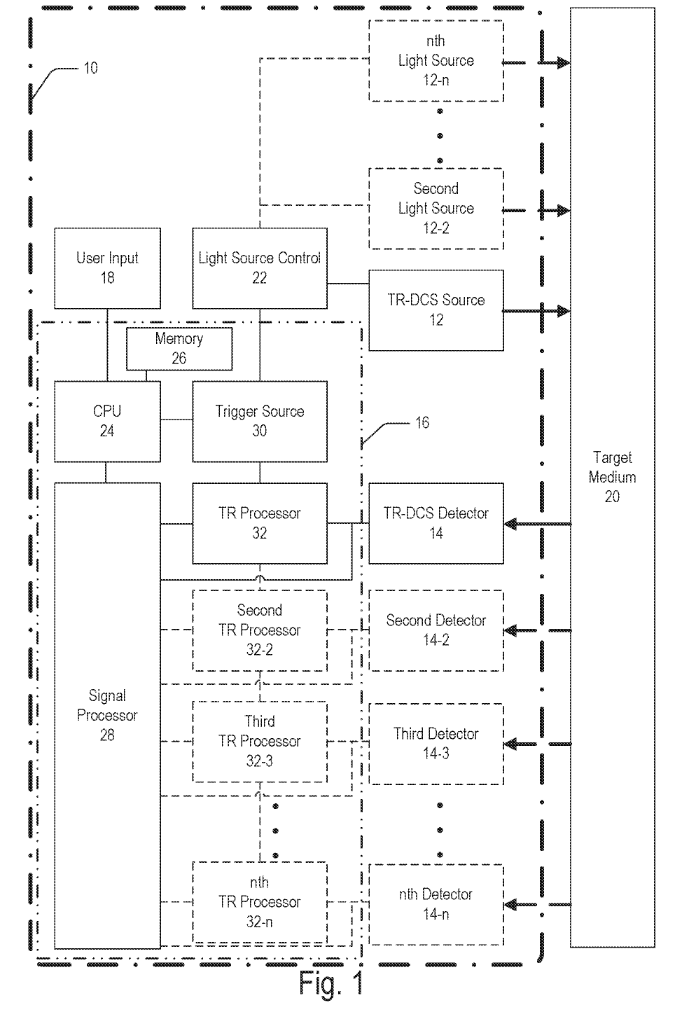

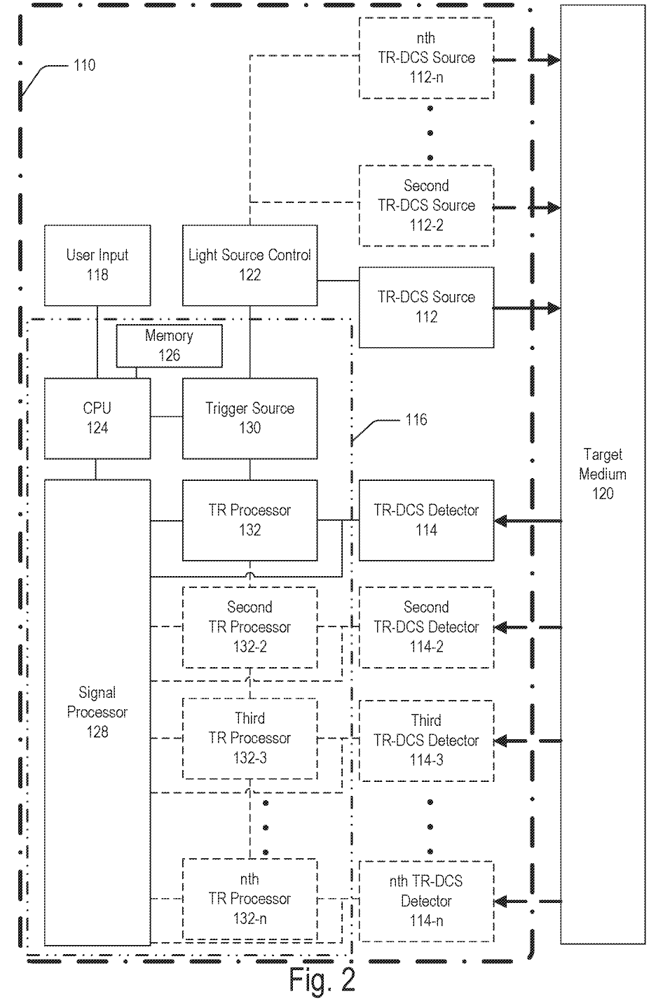

Referring to FIGS. 1 and 2, a system 10, 110 suitable for executing the methods of the present disclosure is provided. The system 10, 110 can include a TR-DCS source 12, 112 and a TR-DCS detector 14, 114. The system 10 can include a computer 16, 116 in electronic communication with the TR-DCS source 12, 112 and the TR-DCS detector 14, 114. The system 10, 110 can also include a user input 18, 118 configured to provide an interface between a user and the computer 16, 116 and/or other aspects of the system 10, 110 (connections between the user input 18, 118 and the other aspects are not illustrated, but can be appreciated by a person having ordinary skill in the art). The TR-DCS source 12, 112 and the TR-DCS detector 14, 114 can be coupled to a target medium 20, 120.

The TR-DCS source 12, 112 can be a light source that is capable of emitting optical signals having the properties described elsewhere in the present disclosure. The TR-DCS source 12, 112 can be a transform, or nearly-transform, limited picosecond pulsed source or a non-transform limited picosecond pulsed source. As used herein, reference to “picosecond” pulses or pulsed source refers to pulses having a pulse width between 1 ps and 10 ns. The TR-DCS source 12, 112 can be a Bragg reflector laser, a distributed Bragg feedback laser, a gain-switched distributed Bragg reflector laser, an external cavity laser, a gain-switched laser, a current pulsed laser, a mode-locked laser, a q-switched laser, combinations thereof, and the like. The TR-DCS source 12, 112 can be a diode laser, a solid-state laser, a fiber laser, a vertical cavity surface-emitting laser (VCSEL), a Fabry-Perot laser, a ridge laser, a ridge waveguide laser, a tapered laser, a master oscillator power amplifier (MOPA) laser, or other type of laser. In certain aspects, the TR-DCS source 12, 112 can be a swept source light source.

The TR-DCS source 12, 112 can emit light that is pulsed, sinusoidally modulated, step modulated, triangularly modulated, and/or arbitrarily modulated.

In aspects where the TR-DCS source 12, 112 is a swept source light source and the emitted light is modulated, the modulation described herein can be amplitude modulation and/or can be sweeping the source. The modulation can sweep the wavelength of the source.

The TR-DCS source 12, 112 can be configured to transmit light into the target medium 20, 120 having a wavelength of between 400 nm and 1500 nm, including but not limited to, a wavelength of between 600 nm and 1000 nm, a wavelength of between 690 nm and 900 nm, a wavelength of between 450 nm and 750 nm, a wavelength of between 500 nm and 1250 nm, a wavelength of between 800 nm and 1350 nm, a wavelength of between 1000 nm and 1400 nm, or a wavelength of between 750 nm and 1450 nm. The TR-DCS source 12, 112 can be configured to transmit light into the target medium 20, 120 having an average power of between 10 μW and 10 W, including but not limited to, an average power of between 100 μW and 1 W, between 1 mW and 500 mW, or between 10 mW and 200 mW.

The TR-DCS source 12, 112 can be configured to transmit pulses of light into the target medium having a pulse width of between 1 ps and 10 ns, including but not limited to, a pulse width of between 10 ps and 1 ns, between 50 ps and 700 ps, or between 100 ps and 500 ps. Pulse widths described herein refer to full-width at half maximum pulse widths.

In certain aspects, a TR-LDF source can be configured to transmit pulses of light into the target medium having a pulse width of between 100 fs and 700 ps.

The TR-DCS source 12, 112 can be configured to transmit pulses of light into the target medium 20, 120 having a pulse repetition rate of up to 1 GHz, including but not limited to a frequency of between 1 kHz and 1 GHz, between 100 kHz and 500 MHz, or between 10 MHz and 400 MHz.

The TR-DCS source 12, 112 can be configured to transmit pulses of light into the target medium 20, 120 having a coherence length that is of the same order of magnitude as the pathlength distribution width of the pulses of light travel through the target medium 20, 120. The TR-DCS source 12, 112 can be configured to transmit pulses of light into the target medium 20, 120 having a coherence length of less than the pulse width times the speed of light in the target medium 20, 120. The TR-DCS source 12, 112 can be configured to transmit pulses of light into the target medium 20, 120 having a coherence length of between 0.01 mm and the transform limit, including but not limited to, a coherence length of between 0.3 mm and 3000 mm, between 3 mm and 300 mm, between 15 mm and 210 mm, or between 30 mm and 150 mm.

In certain aspects, the TR-DCS source 12, 112 can be configured to modulate the pulse width at a modulation frequency from a minimum pulse width to a maximum pulse width. In certain aspects, the modulation can include frequency domain modulation. The modulation can have a sinusoidal waveform, a triangular waveform, a step function waveform, a square waveform, asynchronous trigger, time-division multiplexing, and the like. The modulation frequency must be lower than the pulse repetition rate of the pulses of light. The modulation frequency can be between 0.01 Hz and 500 MHz, including but not limited to, a modulation frequency between 0.1 Hz and 10 Mhz, or between 1 Hz and 1 kHz.

Referring to FIG. 1, in certain aspects, the system 10 can further optionally include a second light source 12-2. The system 10 can also optionally include a third light source, a fourth light source, and so on, up to an nth light source 12-n. Light sources included in the system 10 beyond the TR-DCS source 12 are collectively referred to as additional light sources. These additional light sources can have similar properties to the TR-DCS source 12 or can have substantially different properties, and the different combinations and arrangements can have distinct advantages as described herein.

In certain aspects, the second light source 12-2, the third, fourth, up to nth, and/or additional light sources can be the sources listed with respect to the TR-DCS source 12 or can be a laser, a laser diode, an LED, a superluminescent diode, a broad area laser, a lamp, a white light source, and the like.

Referring to FIG. 2, a system 110 is illustrated that optionally includes multiple TR-DCS sources 112, 112-2, . . . , 112-n and multiple TR-DCS detectors 114, 114-2, 114-3, . . . , 114-n.

In the illustrated aspect of FIG. 2, the system 110 includes a first TR-DCS source 112 and a second TR-DCS source 112-2. It should be appreciated that the system 110 can include a third TR-DCS source, a fourth TR-DCS source, a fifth TR-DCS source, and so on, up to the nth TR-DCS source 112-n. Aspects of the present disclosure described with respect to one TR-DCS source 112, 112-2, . . . , 112-n are applicable to any number of TR-DCS sources 112, 112-2, . . . , 112-n that are contained within the system 10. A person having ordinary skill in the art will appreciate that the number of TR-DCS sources 112, 112-2, . . . , 112-n is not intended to limited in this disclosure, and the number exemplified by the illustrated aspects are specific only for ease of explanation and brevity.

For clarity, this disclosure explicitly contemplates any number of TR-DCS sources 12, 112, 112-2, . . . , 112-n and TR-DCS detectors 14, 114, 114-2, 114-3, . . . , 114-n up to 2, up to 5, up to 10, up to 25, up to 50, up to 100 or more, and up to n TR-DCS sources 12, 112, 112-2, . . . , 112-n and/or TR-DCS detectors 14, 114, 114-2, 114-3, . . . , 114-n.

It should be appreciated that the system 110 of FIG. 2 is a specific aspect of the system 10 of FIG. 1, and therefore, any features described with respect to the system 10 of FIG. 1 are applicable to the system 110 of FIG. 2, and vice versa.

What follows is a non-limiting example of the use of the system 110 illustrated in FIGS. 1 and 2. In this aspect, one or more laser sources produce nearly-transform limited pulses which are directed onto a specimen. Light is received from the specimen by one or more detectors via single-mode or multi-mode optical fibers. Each detected photon is tagged by one or more timestamps. One timestamp represents the time of flight through the tissue and the other represents the time of arrival with respect to a previously detected photon or absolute time. Other aspects may use a single timestamp to record both the time of flight and arrival time. In this aspect, histograms of the times of flight are used to estimate μₐ and μₛ′. In certain aspects, the correlation function and the decay rate slope can be used to calculate μₛ′. These coefficients can be used to estimate flow, and optionally, hemoglobin concentrations and/or blood oxygenation, and result in improved accuracy, precision, and reduced variability with respect the prior art. The intensity correlation function is calculated from the arrival time tag. The correlation functions can be autocorrelation functions calculated from individual detectors, autocorrelation functions calculated from multiple detectors, cross-correlation functions calculated between different detectors, or any combination thereof. Photons are separated into one or more groups based on their time of flight. Different intensity correlations are calculated singly or in combination of one or more groups. The analysis of flow and other hemodynamic and metabolic values can be determined independently or through simultaneous global analysis of the timestamps from one or more sources and/or detectors. The results can provide a single average flow or can be divided to provide multiple flows. The results from different groups may represent flow values from different tissue depths. For example, results including all photons result in the conventional DCS result, results including groups of photons with shorter times of flight result in flows from more superficial tissues while groups with photons with longer times of flight result in flows from deeper tissues. This discrimination of signal by tissue depth has not been previously achieved.

In certain aspects, the TR-DCS source 12, 112, the second TR-DCS source 112-2, the third, fourth, fifth, up to nth TR-DCS Source 112-n, or any additional TR-DCS sources, the second light source 12-2, the third, fourth, up to nth, or any additional light sources can include one or more amplifiers to amplify the intensity of the emitted light. For example, the TR-DCS source 12 can be a pulsed and/or modulated laser that has an optical amplifier that amplifies the intensity of the emitted light, but does not change the time-dependent properties of the light. In aspects including the amplifier, the source can be configured in a master oscillator power amplifier (MOPA) configuration.

In certain aspects, the amplifiers can change the time- or frequency-domain properties of the light. For example, the TR-DCS source 12 can include a continuous wave laser or a laser having a pulse length that is longer or shorter than a desired pulse length, and the amplifier itself can be the source of the desired pulse length, or varying a pulse timing between a pulsed seed light source and a pulsed amplifier can be the source of the desired pulse length. For clarity, the TR-DCS source 12 can be configured to emit light having certain properties described elsewhere herein, and those properties can originate from any of the components of the TR-DCS source 12 including the TR-DCS light source and/or the amplifier. For example, a pulsed laser source and a pulsed amplifier can be pulsed out of phase, and a resulting pulse of light can have a pulse profile that is the overlap of the out of phase pulse profiles.

In certain aspects, the second light source 12-2, the third, fourth, up to nth light source 12-n, and/or additional light sources can have properties that are substantially similar to those described with respect to the TR-DCS source 12.

In certain aspects, the second TR-DCS source 112-2, the third, fourth, up to nth TR-DCS source 112-n, and/or additional TR-DCS sources can have properties that are substantially similar to those described with respect to the TR-DCS source 112.

In some cases, the additional light sources or the additional TR-DCS sources can be configured to emit light that is substantially similar to the light emitted from the TR-DCS source 12, 112. In some cases, the additional light sources or the additional TR-DCS sources can be configured to emit light that is suitable for TR-DCS, but having one or more different properties than the TR-DCS source. For example, the TR-DCS source 12 could emit light having a first pulse length and the second light source 12-2 or the second TR-DCS source 112-2 could emit light having a second, different, longer pulse length, which could allow the measurement of different properties. As another example, the TR-DCS source 12 could emit light having a first wavelength and the second light source 12-2 or the second TR-DCS source 112-2 could emit light having a second, different wavelength, which could allow the use of filters or multiplexing schemes to discriminate between signals originating from the respected sources. It should be appreciated that this discrimination can include optical, electronic, or optical and electronic discrimination.

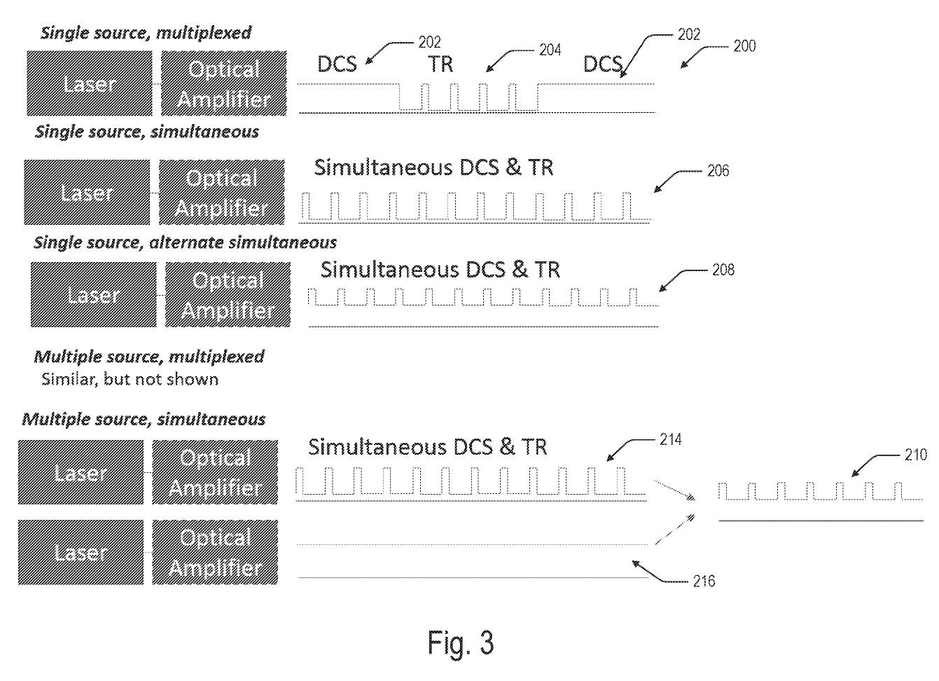

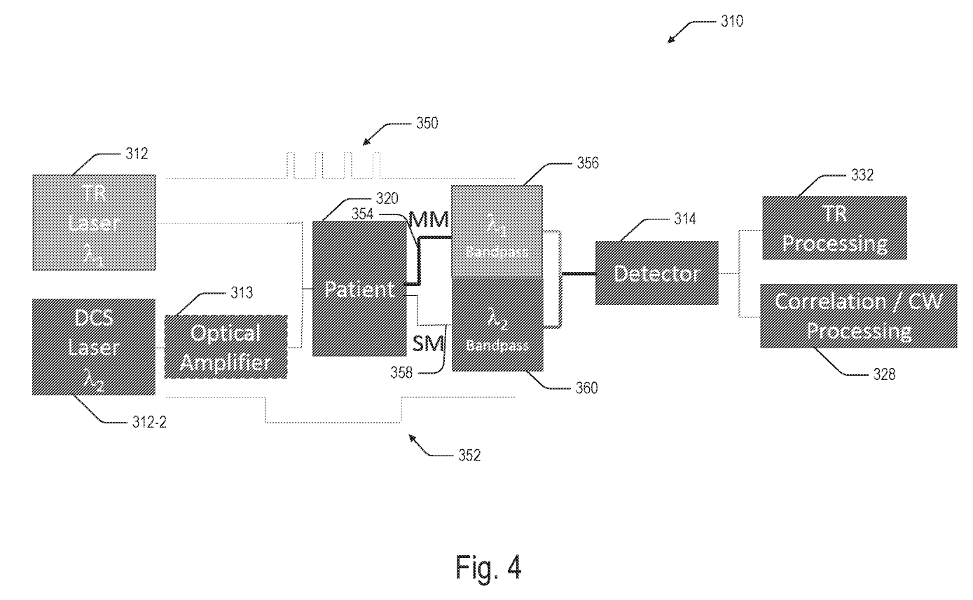

Referring to FIGS. 3 and 4, various example light emission profiles and/or detection schemes are illustrated. A single source, multiplexed emission profile 200 can have a CW portion 202 and a pulsed or modulated portion 204. The single source, multiplexed emission profile 200 can detect and distinguish TRS and DCS signals by time- or frequency-division multiplexing. A single source, with a pulsed emission profile 206 can provide pulsing for TR-DCS measurements. A single source, with a CW and pulsed emission profile 208 can have a continuous component for the DCS measurement with periodic pulses for the TRS measurement. A multiple source, multiplexed pulse profile (not illustrated) can have properties substantially similar to the single-source, multiplexed but is different in that it is formed from two separate emission profiles that are combined. The multiple sources can operate at the same time. A multiple source, simultaneous pulse profile 210 can result from combining a pulsed emission profile 212 and a CW emission profile 214.

Referring to FIG. 4, another example of multiplexing is illustrated. In this configuration, a system 310 can include a time-resolved laser 312 having a first wavelength and emitting a first emission profile 350, a CW laser 312-2 having a second wavelength and emitting a second emission profile 352, an optional amplifier 313, a patient 320 as the target medium, a multimode fiber optic 354 that has a first bandpass filter 356 that passes the first wavelength, a single-mode fiber optic 358 that includes a second bandpass filter 360 that passes the second wavelength, and a detector 314 configured to receive light from both the multimode fiber optic 354 and the single-mode fiber optic 358. Signals from the detector can then proceed to combined or separate TR processing 332 and/or correlation/CW processing 328. The illustrated emission profiles 350, 352 can be multiplexed to switch between different measurement modalities. Other aspects can use a single or multiple light sources, at the same or different wavelengths, and an optical or mechanical switch.

It should be appreciated that the optical amplifiers, the waveguides, the filters, and/or the processors illustrated in FIGS. 3 and 4 are optional, as discussed elsewhere herein. It should also be appreciated that the aforementioned emission profiles can be combined in various ways, according to methods known to those having ordinary skill in the art. It should also be appreciated that the system 310 of FIG. 4 is a specific aspect of the system 10 of FIG. 1, and therefore, any features described with respect to the system 10 of FIG. 1 are applicable to the system 310 of FIG. 4, and vice versa.

The various sources discussed above can be utilized in any combination of continuous wave, pulsed, or modulated.

Referring to FIGS. 1 and 2, the TR-DCS source 12, the second light source 12-2, the additional light sources, including the nth light source 12-n, the second TR-DCS source 112-2, the additional TR-DCS sources, including the nth TR-DCS source 112-n, can be controlled by a light source control 22, 122. The light source control 22, 122 can be configured to interface between the computer and the TR-DCS source 12, 112, the second light source 12-2, the second TR-DCS source 112-2, and the additional light sources/TR-DCS sources to provide control of the various operational parameters of the light sources described elsewhere herein. The light source control 22, 122 can include a light source driver to control the time-dependent properties of the light emitted from the various light sources.

The light source driver can be configured to receive a trigger signal and control the TR-DCS source 12, 112 and any additional TR-DCS sources to emit light pulses with known timing relative to the trigger signal. In aspects utilizing non-DCS time-resolved spectroscopy (TRS) measurements, the light source driver can be configured to receive a trigger signal and control any additional light sources that are TRS sources to emit light pulses with known timing relative to the trigger signal.

In certain aspects, the light source control 22, 112 can be a component of the computer 16, 116. In certain aspects, the light source control 22, 122 can be a standalone component or multiple standalone components. One light source control 22, 122 can control all or some of the various light sources or each of the various light sources can have its own light source control 22.

The TR-DCS detector 14, 114 can be a light detector that is capable of detecting optical signals having the properties described elsewhere in the present disclosure. The TR-DCS detector 14, 114 can be an avalanche photodiode detector, such as a single-photon avalanche photodiode detector, a photomultiplier tube, a Si, Ge, InGaAs, PbS, PbSe, or HgCdTe photodiode or PIN photodiode, phototransistors, MSM photodetectors, CCD and CMOS detector arrays, silicon photomultipliers, multi-pixel-photon-counters, spectrometers, and the like. In certain aspects, the TR-DCS detector 14, 114 can be enhanced to be sensitive to a specific wavelength of light. In certain aspects, the TR-DCS detector 14, 114 can function as a monitor photodiode. In certain aspects, the TR-DCS detector 14, 114 can be a multi-pixel photo-detector that can be utilized to obtain many parallel detection channels on a single detector. In certain aspects including such a detector, a smaller pixel size can increase the DCS contrast. The TR-DCS detector 14, 114 can be analog or photon counting.

The TR-DCS detector 14, 114 can provide a detector signal that can be analog, digital, photon-counting, or any combination thereof.

Referring to FIG. 1, in certain aspects, the system 10 can further optionally include a second detector 14-2 and optionally a third detector 14-3. The system 10 can also optionally include a fourth detector, a fifth detector, and so on, up to an nth detector 14-n. Detectors included in the system 10 beyond the TR-DCS detector 14 are collectively referred to as additional detectors. These additional detectors can have similar properties to the TR-DCS detector 14 or can have substantially different properties, and the different combinations and arrangements can have distinct advantages as described herein.

Referring to FIG. 2, in certain aspects, the system 10 can include a first TR-DCS detector 114, a second TR-DCS detector 114-2, and a third TR-DCS detector 114-3. It should be appreciated that the system 10 can include a fourth TR-DCS detector, a fifth TR-DCS detector, a sixth TR-DCS detector, and so on, up to an nth TR-DCS detector 114-n. A person having ordinary skill in the art will appreciate that the number of TR-DCS detectors 114, 114-2, 114-3, . . . , 114-n is not intended to be limited in this disclosure, and the number exemplified in the illustrated aspects are specific only for the purposes of ease of explanation and brevity. The second TR-DCS detector 114-2, the third TR-DCS detector 114-3, the fourth, fifth, up to nth, and/or additional TR-DCS detectors can be the detectors listed with respect to the TR-DCS detector 14, 114.

In certain aspects, the second detector 14-2, the third detector 14-3, the fourth, fifth, up to nth, and/or additional detectors can be an avalanche photodiode detector, such as a single-photon avalanche photodiode detector, a photomultiplier tube, a Si, Ge, InGaAs, PbS, PbSe or HgCdTe photodiode or PIN photodiode, phototransistors, MSM photodetectors, CCD and CMOS detector arrays, silicon photomultipliers, multi-pixel-photon-counters, and the like, or other optical detectors known to those having ordinary skill in the art. In certain aspects, the second detector 14-2, the third detector 14-3, the fourth, fifth, up to nth, and/or additional detectors can be analog or photon counting.

In certain aspects, the TR-DCS detector 14, 114, the second detector 14-2, the third detector 14-3, the fourth, fifth, up to nth detector 14-n, or any additional detectors, the second TR-DCS detector 114-2, the third TR-DCS detector 114-3, the fourth, fifth, up to nth TR-DCS detector 114-n, or any additional TR-DCS detectors can be configured to receive optical signals from a single location or from multiple locations. Any combination of DCS, TRS, and CW detection can be achieved with the same or different detectors, including various combinations of detectors.

The system 10, 110 can optionally further include waveguides to couple the TR-DCS source 12, 112, the TR-DCS detector 14, 114, the additional light sources, and/or the additional detectors to the target medium 20, 120. The optional waveguides can be any waveguide suitable for delivering light having the properties described elsewhere herein. For example, the optical waveguides can be a fiber optic or a fiber optic bundle, a lens, a lens system, a hollow waveguide, a liquid waveguide, a photonic crystal, combinations thereof, and the like. It should be appreciated that the TR-DCS source 12, 112, the TR-DCS detector 14, 114, the additional light sources, and/or the additional detectors can be directly coupled to the target medium 20, 120.

In certain aspects, the waveguides can be deployed in a probe, including as many waveguides as is practical. In certain aspects, the probe can be affixable to a head of a subject. In certain aspects, the probe can be configured to provide multiple distinct source-detector distances. In certain aspects, the waveguides can be deployed in a catheter.

The various detectors 14, 114, 14-2, 114-2, 14-3, 114-3, 14-n, 114-n can have intervening optics and/or pin hole(s), holograms, and/or detector active area dimensions. The various detectors 14, 114, 14-2, 114-2, 14-3, 114-3, 14-n, 114-n can be used singly, multiply, arrayed, or in any combination.

In certain aspects, the detectors 14, 114, 14-2, 114-2, 14-3, 114-3, 14-n, 114-n can have a small active area (i.e., 0.1 μm to 10 μm) to collect light from one or a few speckles, as can be required for DCS/LDF contrast, or can have a larger active area (i.e., 10 μm to 1 mm), which might not typically be associated with capabilities for DCS/LDF contrast. Combining different detectors with different performance for different modalities can have the advantage of improved overall performance and/or reduction in cost, weight, and/or power consumption. For example, the small active area required for DCS/LDF contrast can limit the maximum distance of the source-detector separation due to the decrease in transmission that is associated with a larger separation. On the other hand, time-resolved and continuous wave detection for non-DCS NIRS do not have this requirement, so detectors with different properties, including but not limited to a larger active area, a lower sensitivity, and the like, could be employed, using the same or different sources, or any combination of the above. Thus, a variety of source-detector separations can be utilized, thus enabling, for example, greater accuracy in determination of scattering and/or absorption coefficients than can be achieved using solely shorter separations. Some aspects have improved cost, weight, and/or power consumption. It should be appreciated that the specific aspects described are not intended to be limiting, and additional combinations of source or sources, detector or detectors, and distance or distances are possible.

In certain aspects, one or more pulse emission profiles, such as a pulse sequence, can be utilized as a reference pulse emission profile. In certain aspects, a single emission profile can be split into two parts, one part can be passed through the target medium 20, 120, while the other part is delayed, either statically or variably, then the two parts are recombined and detected. One advantage of this arrangement is that the reference pulse enables interference and improved DCS/LDF signal-to-noise. In certain aspects, one pulse emission profile having narrower pulses can be applied to a sample, while another coherent or partially coherent pulse emission profile having pulses of longer duration is used as a reference pulse. The profiles can be combined and detected. One advantage of this arrangement is that the longer pulse duration enables greater interference and improved signal-to-noise. In certain aspects, the reference pulse emission profile can have a reference pulse length of between 1 ps and 100 ns, including but not limited to, a reference pulse length of the pulse lengths described elsewhere herein. It should be appreciated that many other possible reference pulse emission profiles can be combined with many other sample pulse emission profiles, in ways understood to those having ordinary skill in the art.

The system 10, 110 can also include various other optics that a person having ordinary skill in the art would appreciate as being useful for aiding the acquisition of optical measurement. The system 10, 110 can include various lenses, filters, variable attenuators, polarizers, coupling optics, dielectric coatings, choppers (and corresponding lock-in amplification systems), pinholes, modulators, prisms, mirrors, fiber optic components (splitters/circulators/couplers), and the like.

In certain aspects, the TR-DCS detector 14, 114 can be configured to receive optical signals from multiple different waveguides, where the multiple waveguides are a part of an optical path that includes a filter.

In a system using a multiplexed emission profile, the shot noise for a time-resolved portion of the profile can be uncorrelated in a CW measurement, and the shot noise for a CW measurement can be uncorrelated in a time-resolved measurement. Signal-to-noise ratio can be dominated by drops in amplitude and are generally linear, so a multiplexed signal having equal parts time-resolved portion and CW portion can result in approximately 50% drop in signal-to-noise.

The computer 16, 116 can take the form of a general purpose computer, a tablet, a smart phone, or other computing devices that can be configured to control the measurement devices described herein, and which can execute a computer executable program that performs the simulations described herein. The computer 16 can include various components known to a person having ordinary skill in the art, such as a processor and/or a CPU 24, memory 26 of various types, interfaces, and the like. The computer 16 can be a single computing device or can be a plurality of computing devices operating in a coordinated fashion.

The computer 16 can include a signal processor 28, 128 that is programmed to interpret the detected optical signals. In one non-limiting example, the signal processor 28, 128 can process the macroscopic arrival time or correlation time of the photon. For example, the signal processor 28, 128 can be implemented as a counter in a field programmable gate array (FPGA), an application-specific integrated circuit (ASIC), or other logic device.

The system 10, 110 can include a trigger source 30, 130 for proving one or more trigger signals that are utilized to control the time-resolved aspects of the system 10, 110. The trigger source 30, 130 can be located in the computer 16, 116. A trigger signal from the trigger source 30, 130 can be utilized in correlating the various time measurements for the detection of photons with the emission timing. In certain aspects, the trigger source 30, 130 can be the TR-DCS source 12, 112 itself. In certain aspects, the trigger signal can be fully or partially asynchronous to the sources and/or detectors. In certain aspects, a single trigger signal can be used for time of flight and correlation and/or arrival time measurements.

The system 10, 110 can include a time-resolved (TR) processor 32, 132 for processing TR signals from the TR-DCS detector 14, 114. The TR processor 32, 132 can be located in the computer 16, 116. In certain aspects, the TR processor 32, 132 can receive a trigger signal from the trigger source 30, 130 and a TR-DCS detector signal from the TR-DCS detector 14, 114. In certain aspects, the TR processor 32, 132 can output a signal that functions as a time of flight tag. The TR processor 32, 132 can output to the signal processor 28. Examples of suitable TR processors include, but are not limited to, a time-to-digital converter (such as the SPADlab TDC card, available commercially from SPADlab at Politechnico de Milano, Milan, Italy), a time-gating converter, a time-to-analog converter, a direct analog sampling processor, and the like. A person having ordinary skill in the art will appreciate that suitable TR processors 32, 132 are provided by a variety of manufacturers, such as Hamamatsu (Hamamatsu City, Japan), Becker & Hickl GmbH (Berlin, Germany), Texas Instruments (Dallas, Tex.), AMS/ACAM (Styria, Austria), and Picoquant GmbH (Berlin, Germany), among others. The system 10, 110 can optionally include a second TR processor 32-2, 132-2, a third TR processor 32-3, 132-3, a fourth TR processor, a fifth TR processor, a sixth TR processor, and so on, up to an nth TR processor 32-n, 132-n. It should be appreciated that, in certain cases, the function of these additional optional TR processors can be achieved by a single TR processor. In other cases, the optional additional TR processors can be separate, distinct components. In some aspects, the processing associated with the TR processor 32, 132 can include, without limitation, processing in the time-domain, frequency-domain, analog domain, digital domain, or a combination thereof.

In certain aspects, the signal processor 28, 128 and/or the TR processor 32, 132 can be configured to extract measurement from the photon signals by a variety of means, including but not limited to, time-correlated methods, time-to-amplitude converter methods, time-to-digital converter methods, Fourier or other transform methods, heterodyning or homodyning methods, or a combination thereof, with examples including but not limited to, hardware-based extraction, software-based extraction, linear transforms, log transforms, multitau correlation, and combinations thereof.

In certain aspects, the signal processor 28, 128 and/or the TR processor 32, 132 can be used to construct a TPSF from which the scattering and/or absorption coefficients can be estimated. In certain aspects, the signal processor 28, 128 and/or the TR processor 32, 132 can be used to estimate the scattering and/or absorption coefficients from a phase shift of the detected signal relative to the source and the associated AC amplitude, DC amplitude, and/or modulation. The estimations of scattering and/or absorption coefficients can be used in estimation of flow and oxygenation, which can be estimated independently or simultaneously with estimation of the coefficients and/or flow. In certain aspects, the estimation of scattering and/or absorption coefficients can be used to estimate the concentration of species of interest.

The TR-DCS detector 14, 114, the signal processor 28, 128, and/or the TR processor 32, 132 can be configured to utilize time-gating of the measured signals. Accordingly, a duty cycle of less than 100% can be utilized, which can prevent detector saturation and/or discriminate photons by time of flight. Time-gating can be achieved in the analog or digital domain, or both. In existing methods, flow was estimated from all detected photons, and significant effort is required to separate out superficial and deeper flow values. In certain aspect of the present disclosure, photons can be classified as early arriving or late arriving (or other combinations of categories that are relevant to the structure of the target medium 20, 120), then flow can be estimated for the different classifications. The early arriving photons are statistically more likely to have spent more time in superficial layers, and therefore, provide more information about flow in superficial layers. The late arriving photons are statistically more likely to have spent more time in the deeper layers, and therefore, provide more information about flow in deeper layers. In certain aspects, the values utilized for separating the photons into different timing groups can be fixed or dynamic, and can be pre-chosen and/or dynamically calculated or adjusted, including combinations thereof.

A detector signal from one of the detectors can be multiplexed to individual processing paths, such as those discussed below, to be processed for DCS, TRS, and/or CW measurements. This multiplexing can afford efficiency in the processing.

In certain aspects utilizing parallel detection channels, when parallel detection channels must be analyzed separately for DCS, the photon counts can be combined and analyzed together by a single TR processor 32, 132. Parallel detection channels can be correlated individually and then combined before transfer. Parallel detection channels can be correlated individually and then moments or other transforms can be transferred.

The processor and/or CPU 24, 124 can be configured to read and perform computer-executable instructions stored in the memory 26, 126. The computer-executable instructions can include all or portions of the methods described herein.

The memory 26, 126 can include one or more computer readable and/or writable media, and may include, for example, a magnetic disc (e.g., a hard disk), an optical disc (e.g., a DVD, a Blu-ray, a CD), a magneto-optical disk, semiconductor memory (e.g., a non-volatile memory card, flash memory, a solid state drive, SRAM, DRAM), an EPROM, an EEPROM, and the like. The memory can store the computer-executable instructions for all or portions of the methods described herein.

The user interface 18, 118 can provide communication interfaces to input and output devices, which can include a keyboard, a display, a mouse, a printing device, a touch screen, a light pen, an optical storage device, a scanner, a microphone, a camera, a drive, a communication cable, or a network (wired or wireless). The interfaces can also provide communications interfaces to the TR-DCS source 12, 112, the TR-DCS detector 14, 114, and other sources and/or detectors includes in the system 10, 110 and/or used in the methods described herein.

The TR-DCS source 12, 112 and the TR-DCS detector 14, 114 can be controlled by the computer 16, 116. The computer 16, 116 can have stored on it a computer executable program configured to execute such control. The computer 16, 116 can direct the TR-DCS source 12, 112 to emit optical signals that are configured to enter into the layered target medium in a fashion that allows the optical signals to interact with fluid flow in the target medium 20, 120, including an inner region of the target medium 20, 120. This interaction can allow the optical signals to acquire information related to the fluid flow in the inner region. The computer 16, 116 can direct the TR-DCS detector 14, 114 to detect the optical signals that contain the acquired information.

In certain aspects, the system 10, 110 can include an imaging modality or a layer thickness measuring modality for characterizing the target medium 20, 120 and providing additional useful information. Examples of suitable imaging and/or layer thickness measuring modalities can include, but are not limited to, an ultrasound imaging system, a non-imaging ultrasound system configured to transmit and receive a reflected acoustic wave, an MRI imaging system, an x-ray imaging system, a computed tomography imaging system, a diffuse optical tomography imaging system, an optical layer thickness measurement system, combinations thereof, or the like. In other aspects, an ultrasound system could be configured to transmit an acoustic wave for depth-specific modulation of the light. Detecting this modulation in the TR-DCS signal could further aid depth discrimination of the flow and hemoglobin information.

In some aspects, the TR-DCS source 12, 112, the TR-DCS detector 14, 114, the computer 16, 116 of the system 10, 110 and other components of the system 10, 110 described herein, including additional TR-DCS sources and/or additional TR-DCS detectors, can be contained in a single unit that is portable and suitable for point-of-care use. In some aspects, the single unit can be handheld. In some aspects, the computer 16, 116 can be a handheld computing device and the remainder of the system 10, 110 can be contained in a single unit that is portable and/or handheld. In some aspects, the system 10, 110 can be contained in one or more handheld units. In some aspects, the system 10, 110 or various components of the system 10, 110 can be contained in a wearable device.

In some aspects, the TR-DCS source 12, 112, the TR-DCS detector 14, 114, and the computer 16, 116 of the system 10, 110 and other components of the system 10, 110 described herein, including additional TR-DCS sources and/or additional TR-DCS detectors, can be contained in a table-top unit that is suitable for placement on a table-top and can be located appropriately for point-of-care use.

The system 10, 110 can be powered by a power supply that is supplied electricity from a wall outlet or via one or more batteries, either rechargeable or replaceable.

It should be appreciated that various aspects of the system 10, 110 that are illustrated as blocks are shown in this fashion for illustrative purposes, and those blocks can be multiple separate elements or can be combined into single monolithic elements.

One advantage of the system 10, 110 is that both deep and superficial flows can be captured using the same detector, with a single source-detector separation. A reduction in the necessary number of detectors can provide improvements with respect to cost, size, weight, and complexity. It should be appreciated, however, that a second separation detector can be utilized in combination with these features. In these cases, the time gates in concert with the second source-detector separations can improve detection of the signal of interest relative to the use of one separation detector alone. It should also be appreciated that multiple separation detectors can be utilized, with the time gates in concert with the multiple source-detector separations improving detection of the signal of interest relative to the use of one separation detector alone.

Another advantage of the system 10, 110 is that very small, lightweight detector fibers or solid state detectors can be used, and thus bendable probes can be used. In some aspects, the TR-DCS system 10, 110 can utilize the same small fibers or the same solid state components as a source and a detector, thereby reducing the number of fibers or electrical components required in a probe. Smaller probes can be desirable for vulnerable patients, such as infants, placement around surgical and/or wound sites, and for use with other measurement modalities, such as EEG, cranial bolts, and the like. Smaller probes are also advantageous for implantable, chronic, mobile, and/or wearable applications. Additional advantages can include reduced cost, weight, and/or power consumption.

Aspects of the present disclosure discussed below with respect to the methods 400, 500, 600, 700 are applicable to and can be incorporated in the systems 10, 110, 310 described herein. For clarity, if the methods below describe an aspect that a person having ordinary skill in the art would understand as implying the presence of structural features in the systems 10, 110, 310 described above, then this disclosure expressly contemplates the inclusion of those structural features. As a non-limiting example, if the methods below describe focusing light, then a person having ordinary skill in the art would understand that this implies the presence of a focusing lens or a structure that serves the purpose of a focusing lens, such as a concave curved mirror.

### Methods

This disclosure provides methods for using the systems 10, 110 described above, although the methods can optionally be used with other systems not described herein.

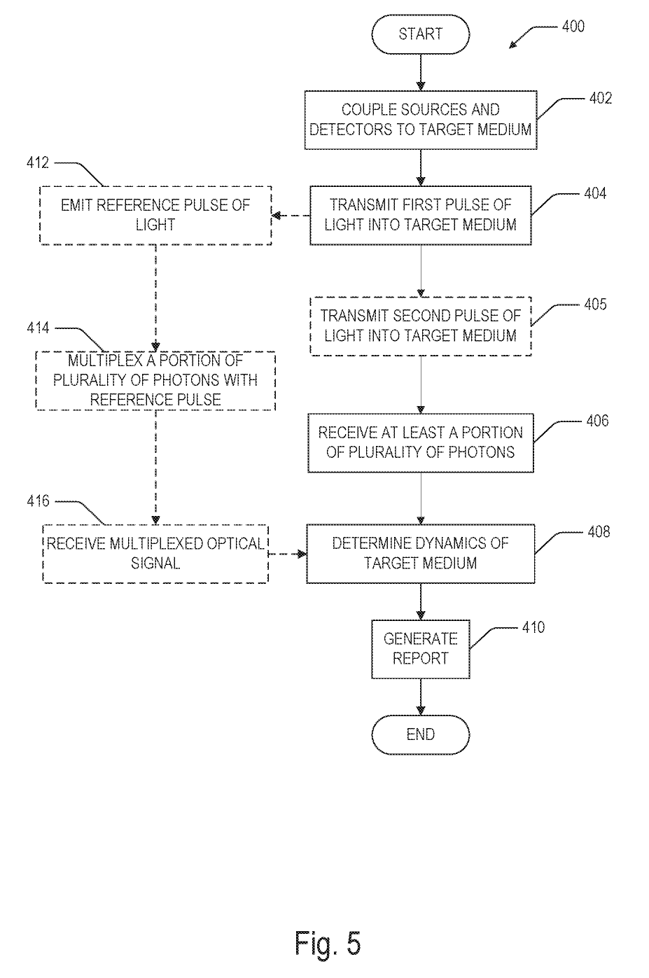

Referring to FIG. 5, this disclosure provides a method 400 for making a time-resolved diffuse correlation spectroscopy measurement of dynamics in a target medium 20, 120. At process block 402, the method 400 can include coupling a TR-DCS source 12, 112 and a TR-DCS detector 14, 114 to the target medium 20, 120. Process block 402 can also include coupling any number of additional sources or detectors to the target medium 20, 120. The TR-DCS source 12, 112 and the TR-DCS detector 14, 114 can have the properties described elsewhere. The TR-DCS source 12, 112 can be configured to emit pulses of light can have a pulse length of between 1 ps and 10 ns. At process block 404, the method 400 can include emitting a first pulse of light from the TR-DCS source 12, 112 into the target medium 20, 120. The first pulse of light can include a plurality of photons. At process block 406, the method 400 can include receiving at least a portion of the plurality of photons at the TR-DCS detector 14, 114 after passing through the target medium 20, 120. The receiving of process block 406 can thereby generate a TR-DCS detector signal including timing information and correlation information for at least a portion of the plurality of photons. At process block 408, the method 400 can include determining dynamics of the target medium. The determining of process block 408 can be executed on a processor or CPU 24, 124, and can utilize the timing information, the correlation information, and one or more equations relating time of flight and correlation to dynamics. The one or more equations can be those discussed below in the “Computational Considerations” section. The dynamics can be depth-dependent dynamics. At process block 410, the method 400 can include generating a report including the dynamics of the target medium 20, 120.

Still referring to FIG. 5, at optional process block 405, the method 400 can include transmitting a second pulse of light from the TR-DCS source 12, 112 or a different source or different TR-DCS source into the target medium. The second pulse of light can include a second plurality of photons. In aspects including optional process block 405, the receiving of process block 406 can include receiving at least a portion of the second plurality of photons at the TR-DCS detector and the generated TR-DCS detector signal can include timing information and correlation information for the at least a portion of the second plurality of photons. The method 400 can include any number of pulses of light and any number of pulse trains from one source or multiple sources. The other methods 500, 600, 700 described herein can also include multiple pulses or multiple pulse trains in a similar fashion.

Still referring to FIG. 5, at optional process block 412, the method 400 can optionally include emitting a reference pulse of light that does not pass through the target medium 20, 120. The reference pulse of light can be emitted by the TR-DCS source 12, 112 or a different light source. At optional process block 414, the method 400 can include multiplexing the at least a portion of the plurality of photons that pass through the target medium with the reference pulse of light, thereby generating a multiplexed optical signal. At optional process block 416, the method 400 can include receiving the multiplexed optical signal at an optical detector, thereby generating a detector signal. The detector signal can be generated via optical heterodyne detection principles understood by those having ordinary skill in the art. The detector signal can include timing information and correlation information for the at least a portion of the plurality of photons, which can be utilized in the determining of process block 408. The optional steps of optional process blocks 412, 414, and 416 can be utilized in the other methods 500, 600, 700 described herein in ways understood to those having ordinary skill in the art.

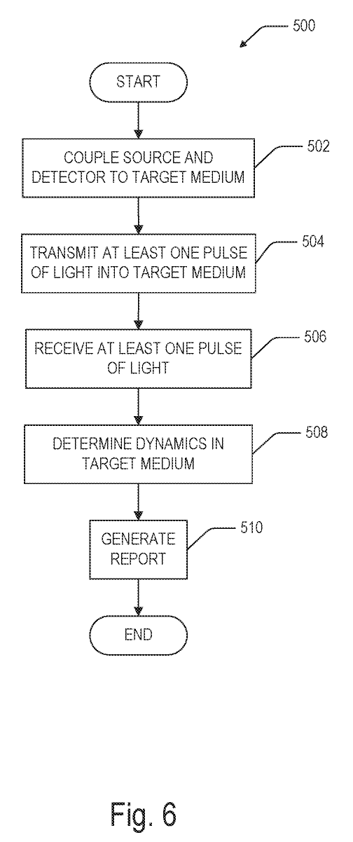

Referring to FIG. 6, this disclosure provides a method 500 for making a time-resolved diffuse correlation spectroscopy measurement of dynamics in a target medium 20, 120. At process block 502, the method 500 can include coupling a TR-DCS source 12, 112 and a TR-DCS detector 14, 114 to the target medium 20, 120. The TR-DCS source 12, 112 and the TR-DCS detector 14, 114 can have the properties described elsewhere. The TR-DCS source 12, 112 can be configured to emit transform-limited, nearly-transform-limited, or non-transform-limited pulses of light. The pulses of light can have a pulse length that is disclosed above with respect to the TR-DCS source 12, 112. At process block 504, the method 500 can include emitting at least one of the pulses of light from the TR-DCS source 12, 112 into the target medium 20, 120. At process block 506, the method 500 can include receiving the at least one of the pulses of light at the TR-DCS detector 14, 114 after the at least one of the pulses of light has traveled through the target medium 20, 120. The receiving of process block 506 can thereby generate a TR-DCS detector signal including depth-specific information about dynamics in the target medium based on the time of flight of the at least one pulse of light. At process block 508, the method 500 can include determining dynamics in the target medium 20, 120. The dynamics can be depth-specific. The determining of process block 508 can be executed on a processor or CPU 24, 124. At process block 510, the method 500 can include generating a report including the dynamics in the target medium 20, 120.

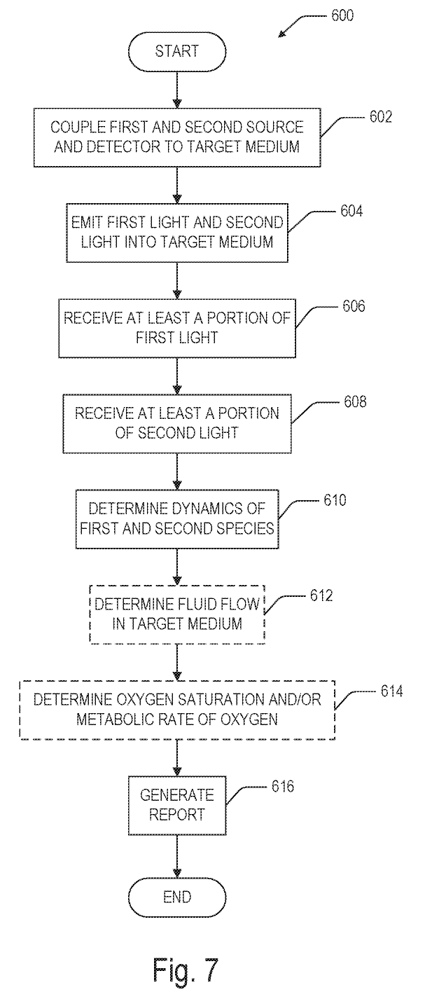

Referring to FIG. 7, this disclosure provides a method 600 of making a TR-DCS measurement of a target medium 20, 120. At process block 602, the method 600 can include coupling a first DCS source, such as a first TR-DCS source 12, 112, a second DCS source 12-2, and a DCS detector 14, 114, to the target medium 20, 120. The first DCS source 12, 112 can be configured to emit a first light comprising first pulses of light having a pulse length of between 1 ps and 10 ns and having a first wavelength. The second DCS source 12-2 can be configured to emit a second light having a second wavelength. At process block 604, the method 600 can include transmitting the first pulses of light from the first DCS source 12, 112 and the second light from the second DCS source 12-2 into the target medium 20, 120. A single source can be used for transmitting the first and second light. The second light can be pulsed light having similar pulse properties to those described elsewhere herein. At process block 606, the method 600 can include receiving at least a portion of the first pulses of light at the DCS detector 14, 114 after the at least a portion of the first pulses of light has traveled through the target medium 20, 120, thereby generating a first DCS signal. At process block 608, the method 600 can include receiving at least a portion of the second light at the DCS detector 14, 114 after the at least a portion of the second light has traveled through the target medium 20, 120, thereby generating a second DCS signal. It should be appreciated that the receiving of process block 606 and 608 can be achieved by separate detectors. At process block 610, the method 600 can include determining dynamics of a first species and a second species in the target medium 20, 120. The determining can use the first DCS signal and the second DCS signal. At process block 616, the method 600 can include generating a report including the dynamics of the first species and the second species.

It should be appreciated that more than two wavelengths of light can be used in the method 600, and other methods 400, 500, 700, and that dynamics and properties can be determined for a corresponding more than two species. Determining the dynamics for a particular number of species can involve use of at least the same number of wavelengths. A person of ordinary skill in the art would appreciate how to solve what can be essentially a linear mixing problem using tools known in the art.

At optional process block 612, the method 600 can optionally further include determining a fluid flow in the target medium. The fluid flow can be determined for each of the first species and second species, or any additional species. The determining of process block 612 can use the first DCS signal and/or the second DCS signal.

In certain aspects, the first species can be oxyhemoglobin and the second species can be deoxyhemoglobin. At optional process block 614, the method 600 can optionally further include determining a hemoglobin, oxyhemoglobin, and/or deoxyhemoglobin concentration, a hemoglobin oxygen saturation and/or a metabolic rate of oxygen. The determining can use the dynamics and/or the fluid flow. The report generated at process block 616 can optionally include the fluid flow, the hemoglobin oxygen dynamics, and/or the metabolic rate of oxygen, either with or in place of the dynamics.

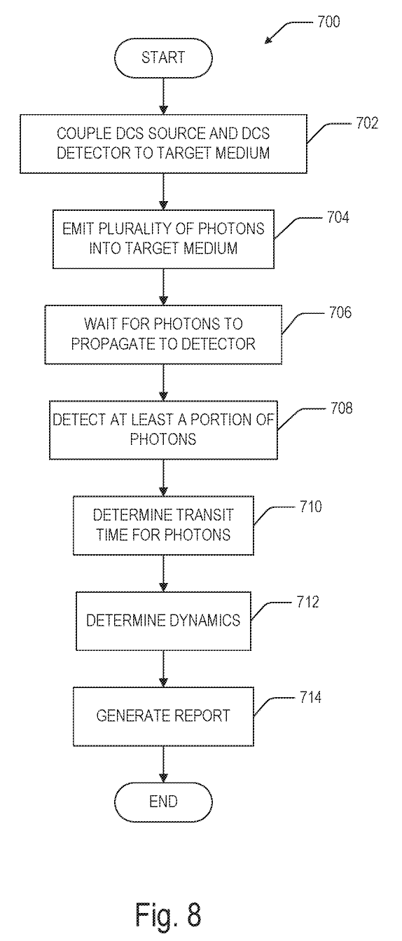

Referring to FIG. 8, this disclosure provides a method 700 of making a time-gated or time-tagged DCS measurement of a target medium. At process block 702, the method 700 can include coupling a DCS source and a DCS detector to a surface of the target medium. At process block 704, the method 700 can include emitting a plurality of photons from the DCS source into the target medium, each emitted photon emitted at a known emission time. At process block 706, the method 700 can include waiting a length of time for at least a portion of the plurality of photons to propagate through the medium from the DCS source to the DCS detector. At process block 708, the method 700 can include detecting the at least a portion of the plurality of photons using the DCS detector, each detected photon detected at a known detection time. At process block 710, the method 700 can include determining a transmit time for each of the at least a portion of the plurality of photons. The determining of process block 710 can include subtracting the known emission time from the known detection time. At process block 712, the method 700 can include determining inner dynamics of an inner portion of the target medium or superficial dynamics of a superficial layer of the target medium, the inner portion and superficial layer defined relative to a surface of the medium. Determining the fluid flow of the inner portion can use photons where the transit time exceeds a pre-determined threshold or a gate time. Measuring the fluid flow of the superficial layer can use photons where the transit time is less than the pre-determined threshold or the gate time. At process block 714, the method 700 can include generating a report including the fluid flow of the inner portion or the superficial layer.

The determining of process blocks 508, 610, 612, 614, 710, and 712 can include calculating using one or more of the equations or concepts described herein. The determining of process blocks 508, 610, 612, 614, 710, and 712 can include fitting data in ways known to those having ordinary skill in the art. The determining of process blocks 508, 610, 612, 614, 710, and 712 can be executed on a processor or CPU 24, 124.

The generating a report of process blocks 410, 510, 616, and 714 can include generating a printed report, displaying results on a screen, transmitting results to a computer database, or another means of reporting the mathematically modeled fluid flow, as would be apparent to a person having ordinary skill in the art. The method 100 is not intended to be limited to a specific report generation.

In certain aspects, the dynamics that are determined by the methods described herein can be fluid flow, shear flow, diffusional properties, motion, association, dis-association, aggregation, dis-aggregation, and/or rotational dynamics of the optical scattering particles within the target medium, and the like.

In certain aspects, dynamics and/or fluid flow can be determined from a group of photons by calculating the correlation function from the arrival times of the photons in the group. Other aspects can utilize other means of measuring dynamics and/or fluid flow, including but not limited to, power spectrum analysis, moment analysis, and the like. The analysis can be performed singly, and/or independently or globally across multiple groups, or combinations thereof. The analysis can be performed by components of the system 10, 110 described above that a person having ordinary skill in the art would appreciate as being capable of the analysis.

In certain aspects, the methods 400, 500, 600, 700 described herein can utilize time-gating detection in ways that can be appreciated by those having ordinary skill in the art. For example, time-gating detection can be utilized on the detector to limit detection to photons having a particular time of flight. Limiting the detection to photons having a shorter time of flight can provide information about more superficial portions of the target medium 20, 120. Limiting the detection to photons having a longer time of flight can provide information about deeper portions of the target medium 20, 120.

In certain aspects utilizing time-gating, the time gate can have a time gate width of between a minimum time-gate resolution and a maximum of the entire time-of-flight window, including but not limited to, a time gate width of between 1 ps and 100 ns, between 10 ps and 6 ns, or between 25 ps and 750 ps. The time gate width can be larger, equal to or less than a coherence length of the pulses of light transmitted into the target medium 20, 120 by the TR-DCS source 12, 112. It was surprisingly discovered that a shorter time gate width can provide superior performance and improved signal-to-noise ratio than a longer time gate width. This is contrary to conventional wisdom, because longer time gate widths allow for more photons to be detected. More photons are generally associated with improved signal-to-noise ratio. It was surprisingly discovered that using the shorter time gate widths disclosed herein provided signal-to-noise ratio gains that resulted from better coherence, and the gains at least somewhat offset the losses from the reduced number of photons detected. With shorter time gates, non-interfering pathlengths can be excluded from the data analysis. Thus, only interfering photons contribute to the correlation function, so coherence is increased within the gate, and the resulting signal-to-noise ratio gains can be achieved.

In certain aspects utilizing time-gating, a plurality of time gates can be utilized to provide additional information about the dynamics of the scattering particles that the pulses of light interact with in the target medium 20, 120. The plurality of time gates can have short time gate widths, such that each of the distinct time gates still relates generally to similar depths of the target medium 20, 120 (i.e., all relate to superficial tissue or all relate to deep tissue, etc.), but the information acquired by using the distinct time gates can better quantify the dynamic properties of the target medium 20, 120 by, for example, comparing the measured decay to the path length.

In certain aspects, the methods described herein can utilize gated detection that involves deactivating a gated detector during an initial time period and activating the gated detector during a subsequent time period. This deactivation can help reduce or eliminate saturation that can result from an initial burst of light arriving at the gated detector.

In certain aspects, the methods described herein can utilize time-resolved detection to perform pulsatile measurements. For example, the methods described herein can measure pulsatile flow, absorption, and/or scattering. This measurement can be synchronized with other physiological measurements, such as blood pressure and/or electrocardiogram measurements. An example of a suitable pulsatile measurement technique can be found in a commonly-owned international patent application entitled “System and Method for Non-Invasively Monitoring Intracranial Pressure”, which claims priority to U.S. Provisional Patent Application No. 62/145,104, and is filed with a docket number of 125141.01531.MGH23304.03, the entire contents of which are incorporated herein by reference.

In certain aspects, the timing information of the methods described herein can include a time of flight tag for each detected photon. In certain aspects, the correlation information can include an arrival tag for each detected photon. The methods can include selecting a subset of detected photons based on the time of flight tag falling within a pre-determined range. Determining steps can then utilize information relating to just the subset of detected photons. The pre-determined range can span a maximum value that is equal to or less than a coherence length of the TR-DCS source. The pre-determined range can be between 1 ps and 100 ns, including but not limited to, between 10 ps and 6 ns, or between 25 ps and 750 ps. The methods can further include selecting a second, third, fourth, or up to nth subset of detected photons based on the time of flight tag falling with distinct pre-determined ranges. The distinct pre-determined ranges can have some overlap or no overlap.

In certain aspects, the methods described herein can utilize measurement at two, three, four, five, six, or more, up to n source-detector distances. Use of multiple source-detector distances can provide better discrimination between various different depths of measurement, such as between cerebral and extra-cerebral measurements. When using multiple source-detector distances, the determinations of the methods can compensate for differences in the timing information and/or time of flight that result from the different source-detector distances.

In certain aspects, the methods described herein can utilize multiple photon time delays for TRS and DCS. Use of multiple photon time delays can provide better discrimination of cerebral and extra-cerebral measurements than can be achieved via CW DCS, and the sensitivity to cerebral blood flow can be increased.

In certain aspects, the methods described herein can utilize two or more different wavelengths of light. Use of two or more different wavelengths of light can afford determination of dynamics for two or more different species. The two or more different wavelengths can afford better quantification of flow, absorption and scattering coefficient measurements, and quantification of hemoglobin concentrations and/or hemoglobin oxygen saturation, which in combination with cerebral blood flow, can provide a measure of CMRO₂. Global analysis can be used to simultaneously determine the flow and hemoglobin concentrations and/or oxygen saturation.

In certain aspect, the methods described herein can combine TR-DCS with CW and time-domain or frequency-domain NIRS.

In certain aspects, the methods described herein can measure properties of the target medium 20, 120 in a baseline state, in a state of spontaneous change, in an evoked change, or a combination thereof. Comparing the measurement of a property following an evoked change with a measurement at a baseline state can provide information regarding the evoked change.

In certain aspects, the methods described herein can utilize detected signals from a single site or multiple sites.

In certain aspects, the correlation described herein can be normalized or unnormalized.

In certain aspects, only a portion of a time-domain histogram can be analyzed. For example, when measuring properties of a deeper portion of the target medium 20, 120, only the later portion of the time-domain histogram may be analyzed. As another example, many small portions of a time-domain histogram (consecutive or partially overlapping) can be analyzed.

In certain aspects, the methods described herein can include a frequency-domain DCS (FD-DCS) measurement. The FD-DCS measurement can utilize the harmonic content of a pulsed laser or a modulated light source.

In certain aspects, the methods described herein can measure the optical properties of the target medium 20, 120 at the same wavelength and in the same location. The measured properties can be used to reduce intra- and inter-subject variability due to anatomy and physiology. In addition, simultaneous, co-localized measurement of DCS and non-DCS time-resolved spectroscopy can be acquired at two or more detector positions and distances relative to a common source.

Calculations, separation, and/or discrimination in the methods described herein can be performed in real-time, near real-time, post-processing, or a combination thereof. These operations can be performed continuously, quasi-continuously, and/or continually, or periodically, and/or intermittently or in batches, or any combination thereof. Alerts, alarms, and/or reports can be generated in response to the results. The alerts, alarms, reports, and/or results can be displayed locally and/or remotely transmitted.

In certain aspects, the methods described herein, and in particular, the time gating features thereof, can be utilized to acquire measurements that are sensitive to areas of the target medium 20, 120 that are near the surface, and can be achieved with a greater source-detector separation, whereas previous methods required a short source-detector separation to isolate measurements near the surface. Similarly, the methods described herein, and in particular, the time gating features thereof, can be utilized to acquire measurements that are sensitive to areas of the target medium 20, 120 that are deeper, and can be achieved with a shorter source-detector separation, whereas previous methods requires a long source-detector separation to isolate measurements deeper in the target medium 20, 120. One advantage that a short source-detector separation provides is that a larger number of photons can be measured, thereby improving the signal-to-noise ratio.

The target medium 20, 120 can include an inner region and a superficial layer. The superficial layer can include one, two, three, four, five, six, or more distinct layers. In some aspects, the superficial layer can include two, three, or four distinct layers.

The superficial layer can include a skull of a subject, a scalp of a subject, a fluid layer between the skull and a cerebral region of a subject, or a combination thereof. The inner region can include a cerebral region of a subject.

The fluid can be blood, water, cerebro spinal fluid (CSF), lymph, urine, and the like. The fluid flow can be blood flow, water flow, CSF flow, lymph flow, urine flow, and the like.

In certain aspects, the target medium 20, 120 can be an industrial fluid of interest. In certain aspects, the target medium 20, 120 can be tissue, including but not limited to, mammalian tissue, avian tissue, fish tissue, reptile tissue, amphibian tissue, and the like. In certain aspects, the target medium 20, 120 can be human tissue.

Aspects of the present disclosure discussed above with respect to the systems 10, 110, 310 are applicable to and can be incorporated in the methods described herein. For clarity, if the systems above describe a structural feature that a person having ordinary skill in the art would understand as implying the presence of a method step or feature in the methods described above, then this disclosure expressly contemplates the inclusion of those method steps or features. As a non-limiting example, if the methods above describe a focusing lens that receives a collimated light beam, then a person having ordinary skill in the art would understand that this implies the presence of a method step or feature involving focusing of light.

### Computational Considerations

The decay of the intensity correlation function can be described by a correlation diffusion equation that is similar to the regular photon diffusion equation but replacing the traditional absorption coefficient (μₐ) with a dynamic absorption coefficient. That is, in the traditional photon diffusion equation, μₐ is replaced with the dynamic absorption term μₐ+2μₛ′D_{B}kₒ²τ to obtain the correlation diffusion equation, where μₛ′ is the reduced scattering coefficient, D_{B} is the Brownian diffusion coefficient acting as an index of blood flow, kₒ=2πn/λ is the wavenumber of light, and τ is the correlation time. Thus, the solution of the time domain-diffuse correlation diffusion equation can be obtained from the traditional TD-NIRS solution by making this replacement. For a semi-infinite medium, the time-domain DCS (TD-DCS) solution for the field auto-correlation function, G₁, is thus:

$${G}_{1} ( τ , ρ , z = 0 , t ) = 2 π {cz}_{b} ( {z}_{0} + 2 {z}_{b} ) {( \frac{3 {μ}_{s}^{′}}{4 π ct} )}^{\frac{5}{2}} exp ( - ( {μ}_{a} + 2 {μ}_{s}^{′} {D}_{B} {k}_{O}^{2} τ ) ct ) exp ( - \frac{3 {μ}_{s}^{′} {ρ}^{2}}{4 ct} ) , \qquad ( 1 )$$

where t is the arrival time of the photons with respect to the laser pulse at t=0, ρ is the source-detector separation.

The normalized field temporal auto-correlation function is obtained by dividing G₁(τ) by G₁(τ=0). Doing so, the path length dependent auto-correlation function is obtained, as follows:

g₁ₛ(τ,S)=exp(−2μₛ′D_{B}kₒ²Sτ), (2)

where the transit time of like through the tissue, t, has been replaced with the path length of light through the tissue, S. This is an important equation as it indicates that the decay rate of the field temporal auto-correlation function increases linearly with the photon path length. Another important result of this equation, is that the path length dependent decay of g₁ₛ(τ,S) is independent of the absorption coefficient of the medium. g₁ₛ(τ,S) was originally derived by first principles and extended to CW-DCS by integrating over the distribution of detected photon path lengths, i.e.

g₁(τ)=∫dsP(s)g₁ₛ(τ,s) (3)

For light diffusion through a highly scattering medium, P(s) is given by the solution of the time-domain photon diffusion equation.

Experimentally, the normalized intensity auto-correlation function (g₂) is measured, which is related to G₁ by g₂=1+β G₁²(τ)/G₁² (τ=0), where β accounts for loss of coherence due to the spatial and temporal coherence of the detected light. DCS signal-to-noise ratio (SNR) is linearly proportional to β. Spatial coherence, and by extension (SNR), is maximized by limiting the detected area, typically by using a single mode fiber to define the detection. The best values of β achieved in conventional DCS is 1 using polarizers or 0.5, without polarizers. For TD-DCS, the coherence length of the pulse of light is generally less than the distribution of photon path lengths through the scattering medium, resulting in a further reduction in β because of reduced temporal coherence at detection. β can be calculated for different pulse lengths to estimate the influence on different pulse parameters and gates on the TD-DCS SNR. For example, β can be estimated from the relation

$${g}_{2} ( τ ) = 1 + \int ds \int {ds}^{′} {g}_{1} ( s , τ ) {g}_{1} ( {s}^{′} , τ ) {e}^{- {2 [ \frac{( s - {s}^{′} )}{{l}_{C}} ]}^{2}} .$$

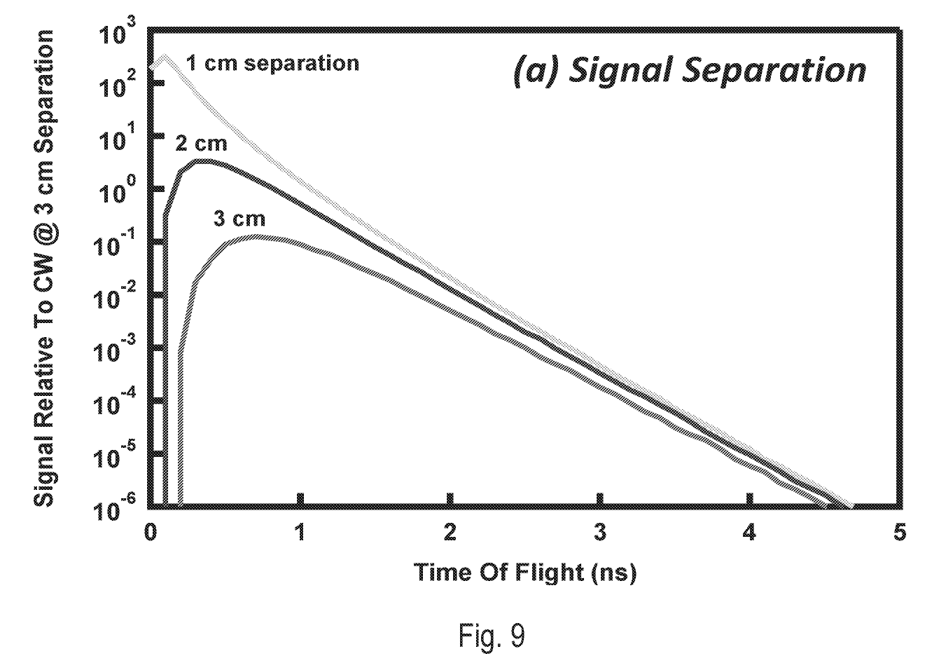

For a source-detector separation of 1 cm, μₐ=0.15 cm⁻¹ and μₛ′=12 cm⁻¹, as we used for the Monte Carlo simulations in FIGS. 9-11, and measuring photons arriving after 1 ns, we find that β is reduced to 0.28 when using a 300 ps pulse of light. We assume that the coherence length of the pulsed light is transform limited and given by the pulse width times the speed of light. Longer laser pulses have longer coherence lengths, larger β, and more photons, but reduce the accuracy with which we can path length resolve the detected light and estimate the scattering coefficient from the TPSF measurements.

The drop in SNR can be overcome, for example, by detecting 3× more photons than acquired in CW-DCS. This increase is practical to achieve by simply using shorter separations, greater laser powers, or longer integration times. Longer laser pulses have longer coherence lengths, larger β, and more photons, but reduce the accuracy and precision with which optical properties can be estimated from TPSF measurements, or equivalent. Thus, by using a modulated or pulsed source, DCS SNR marginally decreases, but this decrease is unexpectedly offset by the benefits of the aspects of this invention, thus producing an unanticipated net increase in performance.

For both DCS and non-DCS time-resolved spectroscopy modalities, measurements at multiple distances facilitate the discrimination of cerebral parameters from the confounding effects of the scalp. For example, by measuring the TPSF at three different distances, intracerebral absorption and scattering parameters can effectively be separated from extra-cerebral ones. These aspects also enable novel strategies for simultaneous TD and DCS, especially including two-layer fitting of the TPSF and DCS to quantify cerebral and extra-cerebral optical properties and blood flow. Extra-cerebral thickness can be estimated through multi-distance DCS measurements. However, the estimation from DCS depends on the absorption and scattering properties of the layers. By combining TD and DCS measurements in this invention, a model consisting of one or more layers can be fit across both modalities and estimate layer thickness, absorption, scattering and blood flow all at once from the data. Thus, this invention is a significant innovation which directly addresses the most fundamental complications of transcutaneous cerebral optical measurements. The measurements and analyses of this invention can be performed with a single source-detector separation, or across multiple distances with multiple detector and/or sources with global or independent analysis, in any combination, in whole or in part.

### EXAMPLES

### Example 1. Monte Carlo Simulation

Using a theoretical apparatus as illustrated in FIG. 2 and the theoretical considerations discussed above, Monte Carlo simulations were performed on a two-layer system to illustrate the results that can be achieved with aspects of the present disclosure. Monte Carlo simulations were also performed for CW-NIRS and CW-DCS. FIGS. 9-11 shows the results of these simulations. The two-layer system had a top layer with thickness of 12 mm and a bottom layer with infinite thickness. The following parameters were used in the simulation: μ_{s,top}′=12 cm⁻¹; μ_{a,top}=0.10 cm⁻¹; BFiₜₒₚ=1×10⁻⁸ cm²/s; μ_{s,bottom}′=12 cm⁻¹; μ_{a,bottom}=0.15 cm⁻¹; and BFi_{bottom}=6×10⁻⁸ cm²/s.

In FIG. 9, the absolute signal as a function of time of flight is shown for three different source detector separations. Typically, a probe is placed non-invasively on the surface of the scalp or skin for transcranial measurement of cerebral blood flow and/or oxygenation. In the CW DCS prior art, source-detector separations of 2 cm or more are typically required to achieve sufficient depth of penetration into an adult head to enable measurement of properties of the brain. In the TR and CW NIRS prior art, typical separations of 3 cm or more are used to reach the brain for cerebral oxygenation measurements. Greater separations are required for NIRS compared to DCS. DCS has an advantage in sensitivity to the brain because the greater blood flow rates in the brain as compared to the scalp enable some discrimination of the desired brain flows from the undesirable superficial flows. As can be seen from FIG. 9, the absolute intensity of transmitted light decreases with time of flight, but increases with smaller source detector separations.

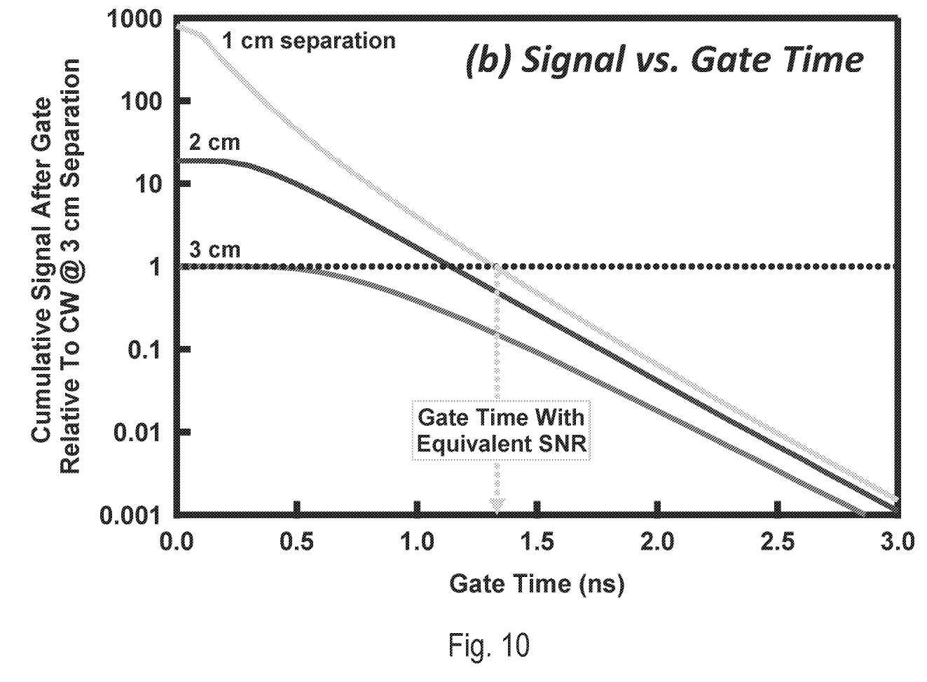

FIG. 10 shows the cumulative total number of photons which arrive later than a time of flight designated as the gate time. The use of later gate times results in signals more representative of deeper tissues, which is highly desirable. However, as the figure shows, later gate times result in lowers signal levels, which is highly undesirable as the signal-to-noise ratios are also correspondingly worse. Thus, there is a practical limit to the amount of time gating which may be employed. For example, from the figure, for a source-detector separation of 3 cm, a typical value used in the prior art, a time gate time of 1.3 ns results in a 90% reduction of signal. It is important to note that in the prior art this 10% of light represents primarily the actual signal of interest, and the 90% of signal before the gate primarily represents undesirable superficial signal. So although the signal rejected by the time gate is large in magnitude, it does not contribute much information of interest. Thus, the actual reduction in signal-to-noise by using a time gate is not as large as naively would be expected. However, another advantage of this invention is that the signal-to-noise ratio can be increased by decreasing the source-detector separation while also improving sensitivity to signals from the brain. This is not possible in the prior art, because in that case decreasing the separation decreases the contribution of the desired signal of interest from the brain. However, a novelty of this disclosure is that a time gate or gates can be employed to select and/or discriminate signals of interest. For example, in FIG. 10, the dashed line shows a time gate applied at 1.3 ns. In this case, the signal arising from a source-detector separation of 1 cm detected after the time gate has a signal-to-noise comparable to the signal-to-noise ratio of the entire signal used in the prior art. In this invention, the signal comes primarily from the tissue of interest, while in the prior art the signal primarily comes from the superficial tissue. Thus, this disclosure enables use of smaller source-detector separations, with the benefits of increased signal and sensitivity to the tissue of interest.

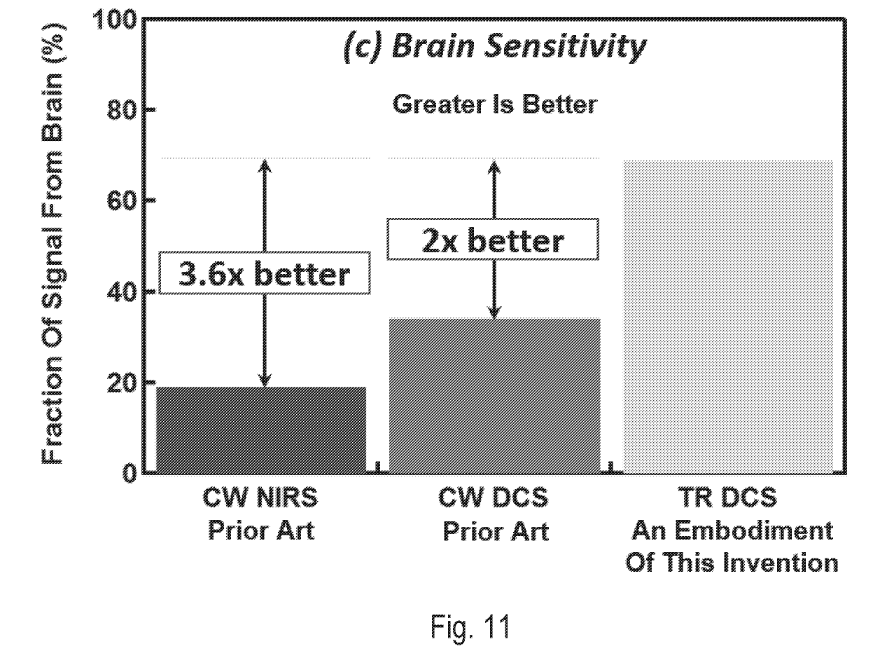

In FIG. 11, a simulation shows a typical net benefit of 2× sensitivity to brain over DCS prior art and 3.6× sensitivity compared to NIRS prior art. Under other configurations, embodiments of this invention may confer even greater improvements.

### Example 2. Static Homogeneous Phantom

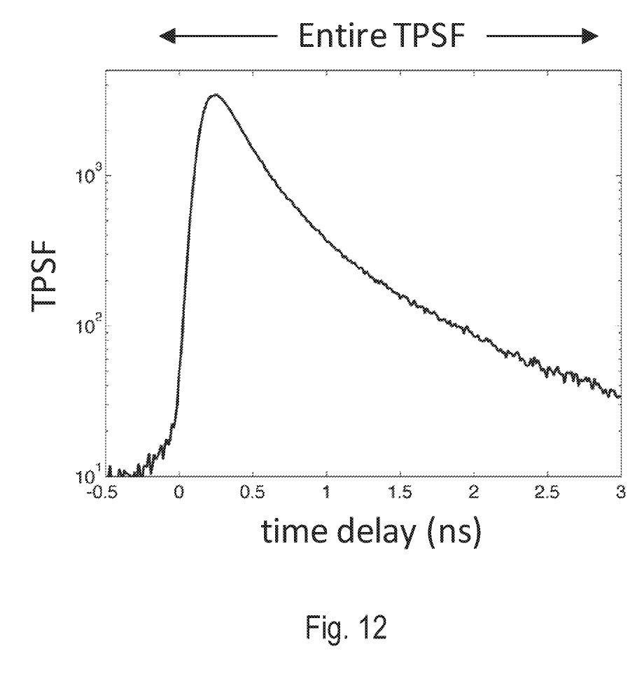

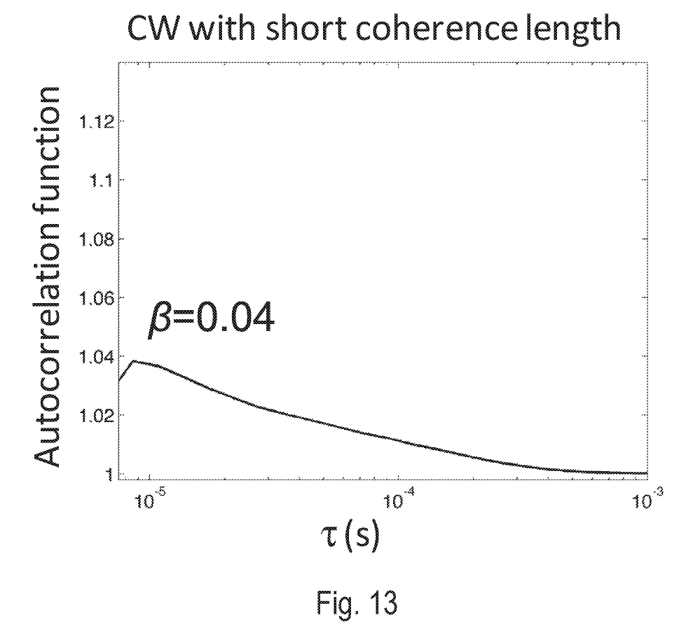

A system similar to that illustrated in FIG. 1 was used for this example. The system included a single TR-DCS source and a single TR-DCS detector separated by 1 cm. The target medium was a diluted milk sample having the following properties: μₛ′=7.5 cm⁻¹; and μₐ=0.04 cm⁻¹. The TR-DCS source was a pulsed laser that emitted 100 ps pulses of light at 150 MHz. As shown in FIG. 12, the TPSF collected by the detector had a pulse length of ˜6 ns. Integration of the entire TPSF provided a correlation function, shown in FIG. 13. This correlation function would be equivalent to a CW correlation function with a short coherence length laser. The amplitude of the autocorrelation function β was reduced, because short and long paths do not interfere. This illustrates an additional factor that had to be taken into account with respect to signal-to-noise ratio.

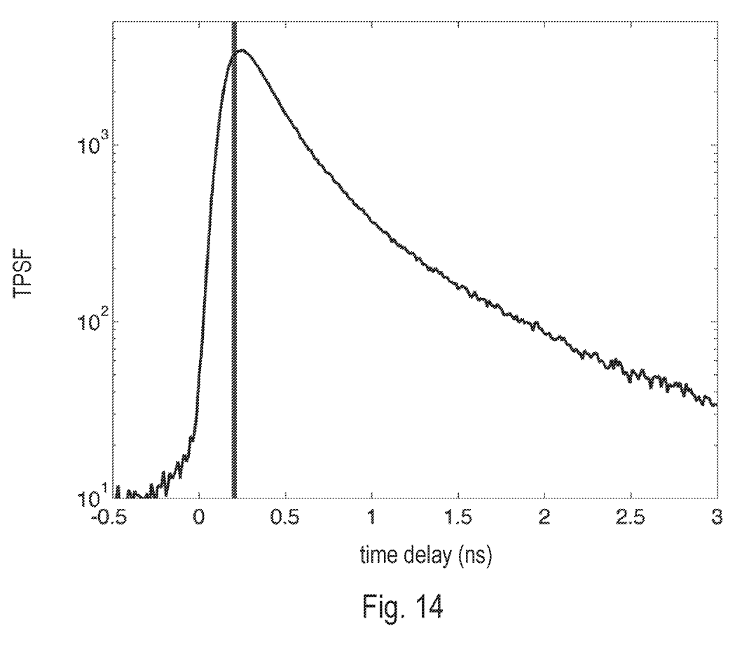

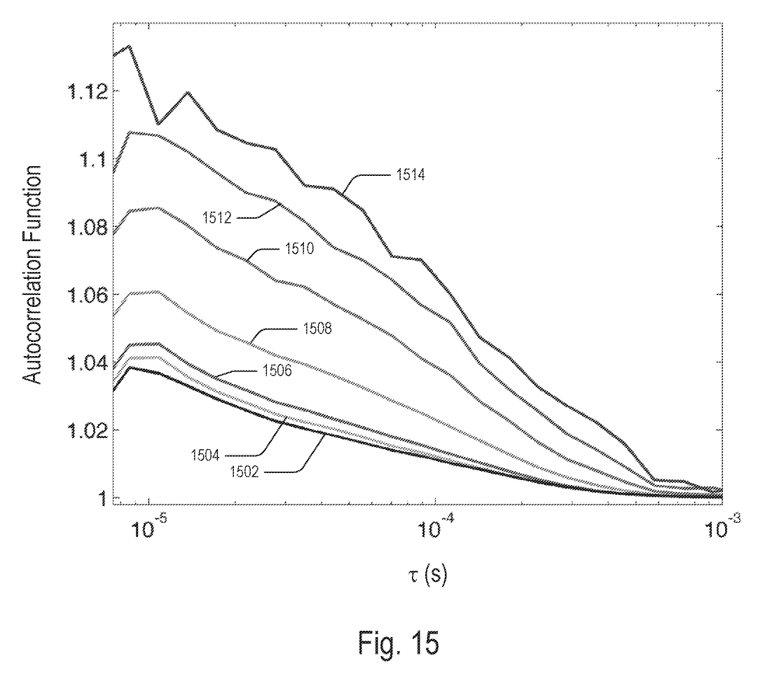

Several gates were applied to the TPSF as follows (reference numbers for FIG. 15 following hyphen): CW (>3 ns)—1502, 720 ps—1504; 480 ps—1506; 240 ps—1508; 120 ps—1510; 60 ps—1512; and 24 ps—1514. The 24 ps gate is illustrated on the TPSF in FIG. 14 and the other gates had the same starting time. The autocorrelation functions are illustrated in FIG. 15, with the reference numbers above pointing to the function for each respective gate. This data illustrates that need to find a compromise between the coherence length of the light source, the TPSF, and the gate width in order to maximize signal-to-noise.

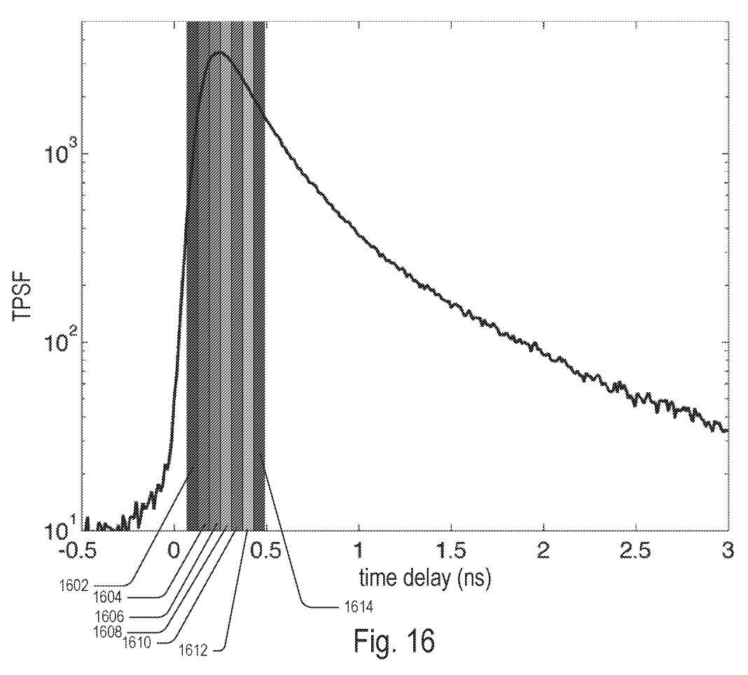

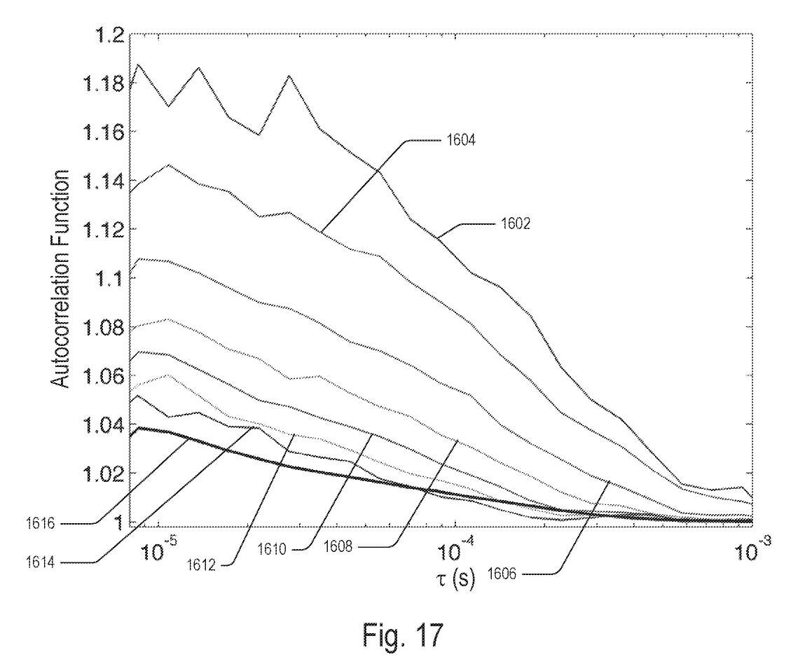

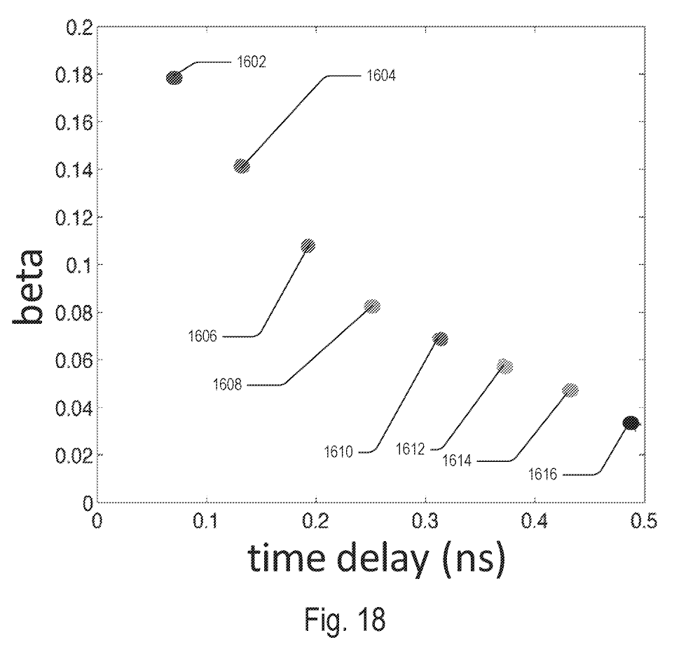

It was further discovered that another factor relevant to signal-to-noise ratio is that the amplitude of the correlation functions, β, also depended on the path length distribution. Using the same arrangement, a set of 60 ps gates were applied to the TPSF at varying times, as illustrated in FIG. 16. The first 1602, second 1604, third 1606, fourth 1608, fifth 1610, sixth 1612, and seventh gate 1614 provided the autocorrelation functions plotted in FIG. 17 and the measured β values plotted in FIG. 18. A CW autocorrelation function and measured β value is represented by reference number 1616.

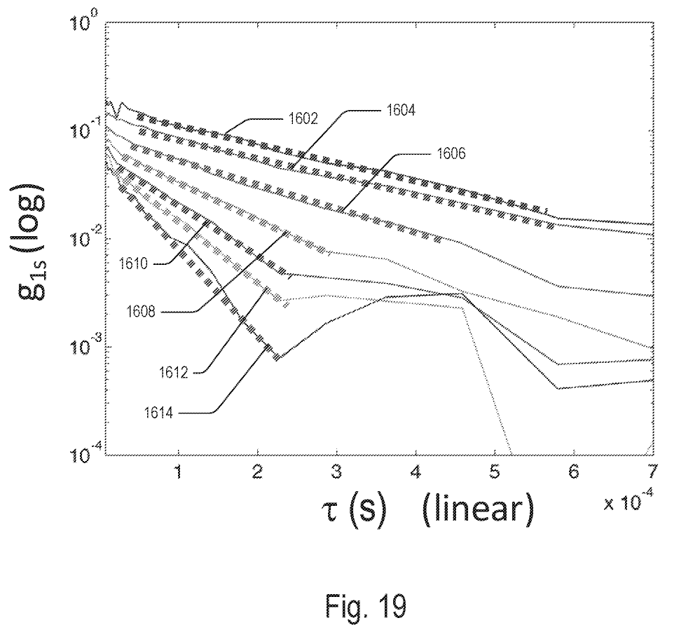

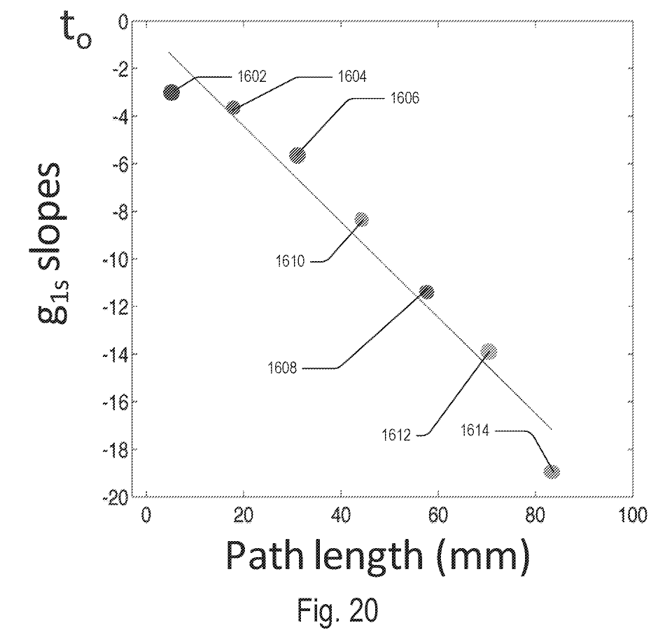

The flow was estimated from the correlation curves by using the equations described above in the Computational Considerations section. The path length was determined from the product of the time gate and the speed of photons in the media. The scattering coefficient can be measured from the TPSF. The path-length-dependent autocorrelation functions for the different time gates were plotted, as illustrated in FIG. 19, and the product of blood flow and the scattering coefficient was subsequently extracted. D_{b}μₛ′s was fit versus s to get D_{b}μₛ′. The resulting slopes from the fit were plotted against the path lengths, as illustrated in FIG. 20. This plot provides a calibration for t₀ for the TPSF. These results confirm the theoretically-predicted linear behavior as a function of path length.

### Example 3. Dynamic Homogeneous Phantom

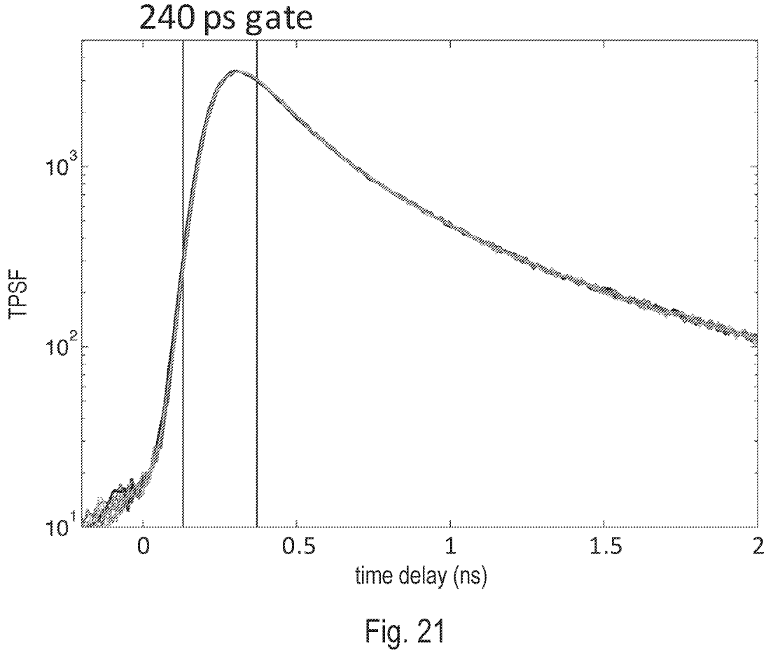

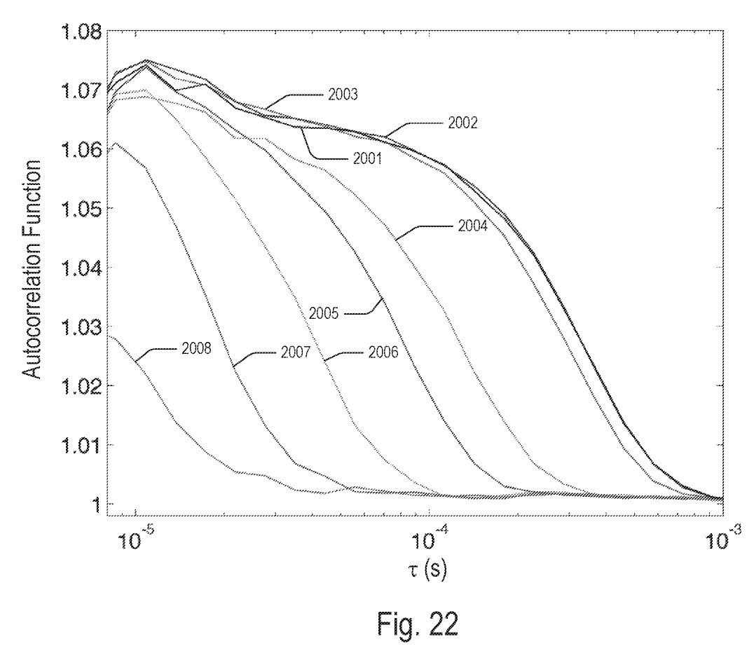

The experimental setup from Example 2 was used in Example 3. The target medium was a silicone solution having the following properties: μₛ′=3.5 cm⁻¹; and μₐ=0.04 cm⁻¹. A stirring mechanism was placed at the bottom of the silicone solution. The TPSF was acquired for a variety of stirrer speed settings 1, 2, 3, 4, 5, 6, 7, and 8, where a larger number indicates faster stirring. The TPSF was identical for each of the different stirrer speeds, as plotted in FIG. 19. A 240 ps gate was applied to the TPSF as shown in FIG. 21. The autocorrelation functions for the different stirrer speeds are plotted in FIG. 22. The reference numbers for stirrer speed settings 1, 2, 3, 4, 5, 6, 7, and 8 are 2001, 2002, 2003, 2004, 2005, 2006, 2007, and 2008, respectively. The autocorrelation function decays faster with increasing stirrer speed, i.e., increasing flow. This example provides evidence of sensitivity to changes in flow.

### Example 4. Time-Gated Flow In Vivo

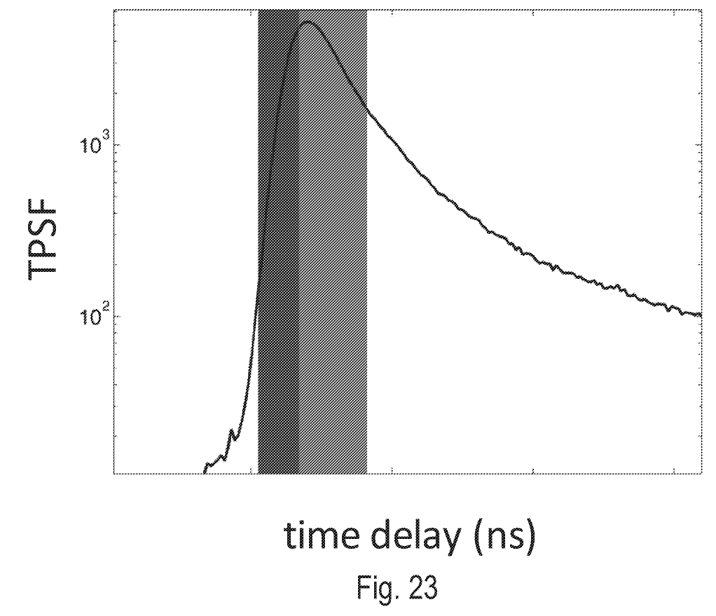

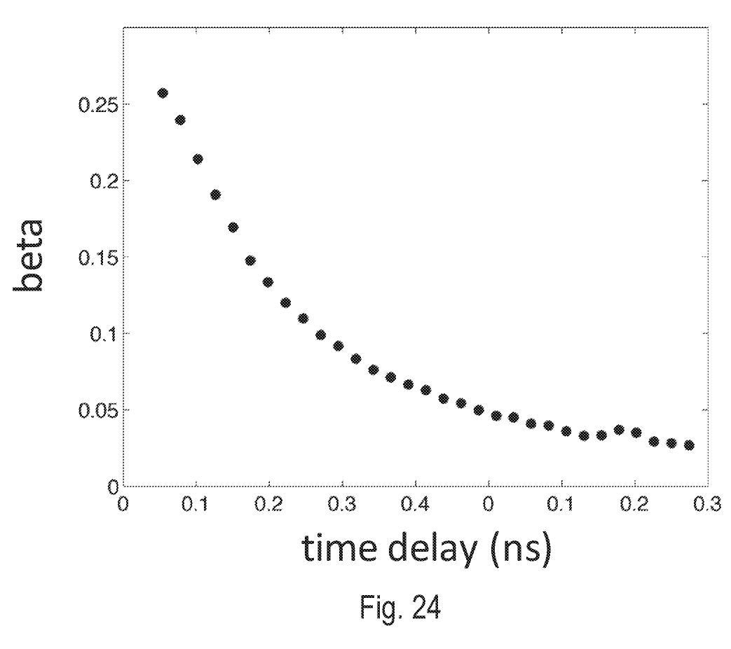

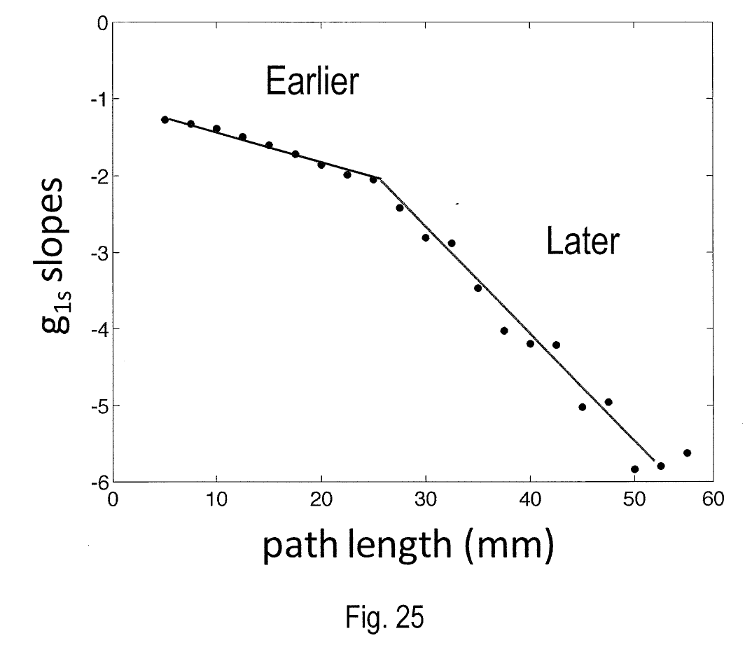

The light source and detector of Examples 2 and 3 were coupled to the head of a rat at a separation distance of 0.5 mm. The TPSF shown in FIG. 23 was acquired and 31 gates were applied to the TPSF. Each gate was 48 ps and they were each shifted 12 ps relative to the previous gate. The gates spanned the highlighted rectangles in FIG. 23. The amplitudes of the time-gated autocorrelation function β were plotted against the time delay, as shown in FIG. 24. Again, the amplitudes follow the predicted behavior. Plotting the slope of g₁ₛ versus the path length, as illustrated in FIG. 25, revealed two different regimes. The shorter path-length regime, denoted “Earlier” in FIG. 25, is for earlier D_{b}μₛ′ and the longer path-length regime, denoted “Later”, is for later D_{b}μₛ′. Assuming that the scattering may be constant, these different slopes can be a product of different flows. The slower slope of the earlier D_{b}μₛ′ corresponds to the slower flow in the scalp and skull region and the faster slope of the later D_{b}μₛ′ corresponds to the faster flow in the cerebral region.

### Example 5. Hypercapnia

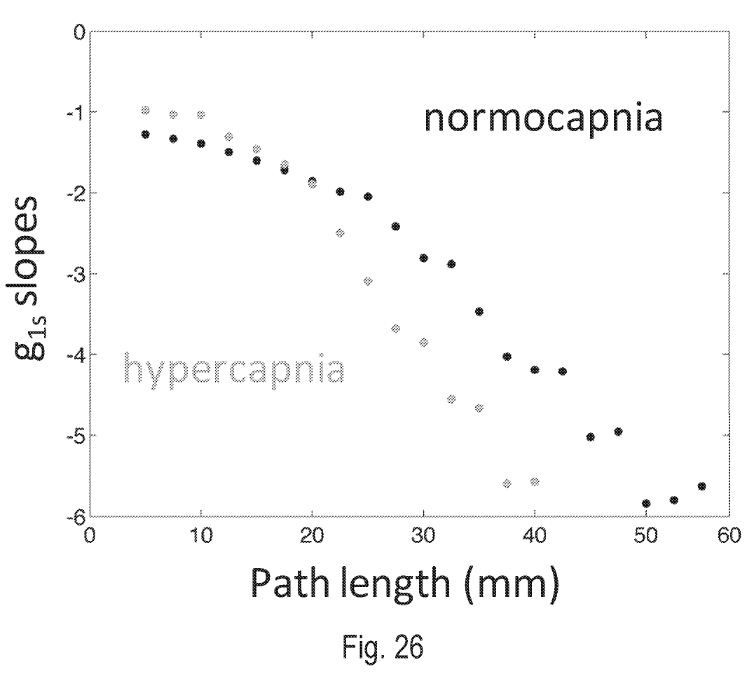

The experimental setup and procedure of Example 4 was repeated with the rat alternately under normal breathing conditions and mechanically ventilated with a few percent CO₂, which has the effect of differentially increasing the blood flow in the brain, without increasing blood flow in the periphery. A plot of the normocapnia and hypercapnia results are shown in FIG. 26. The shorter path length photons do not show a change between normocapnia and hypercapnia, but the later-arriving photons have a faster slope, which is indicative of the faster flow expected in the brain.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the disclosures described herein can be embodied within a form that does not provide all of the features and benefits set forth herein, as some features can be used or practiced separately from others. The scope of certain disclosures disclosed herein is indicated by the appended claims rather than by the foregoing description. All changes which come within the meaning and range of equivalency of the claims are to be embraced within their scope.

We claim:

1. A time-resolved diffuse correlation spectrometry (TR-DCS) system comprising:
a TR-DCS source, the TR-DCS source configured to transmit pulses of light into a target medium, the pulses of light having a pulse length of between 1 ps and 10 ns;
a TR-DCS detector, the TR-DCS detector configured to receive the pulses of light from the target medium and to generate a TR-DCS detector signal in response to receiving the pulses of light;
a memory storing one or more equations relating time of flight and correlation to dynamics of scattering particles within the target medium; and
a processor coupled to the TR-DCS detector and the memory, the processor configured to determine a dynamics of the target medium using the TR-DCS detector signal and the one or more equations.

2. The system of claim 1, the pulses of light having a pulse length of between 10 ps and 700 ps.

3. The system of claim 1, wherein the TR-DCS source is a transform limited or nearly-transform limited pulsed light source and the pulses of light are transform limited or nearly-transform limited pulses of light.

4. The system of claim 1, wherein the TR-DCS source is a Bragg reflector laser, a distributed Bragg feedback laser, a gain-switched distributed Bragg reflector laser, an external cavity laser, a mode-locked laser, a q-switched laser, or a combination thereof.

5. The system of claim 1, wherein the TR-DCS source is a diode laser, a solid-state laser, a fiber laser, of a combination thereof.

6. The system of claim 1, wherein the TR-DCS source is a swept source.

7. The system of claim 1, wherein the TR-DCS source is configured to transmit the pulses of light into the target medium at a wavelength of between 400 nm and 1500 nm.

8. The system of claim 1, wherein the TR-DCS source is configured to transmit the pulses of light into the target medium at an average power of between 10 μW and 10 W.

9. The system of claim 1, wherein the TR-DCS source is configured to transmit the pulses of light into the target medium at a frequency of less than or equal to 1 GHz.

10. The system of claim 1, wherein the TR-DCS source is electronically or optically pulsed.

11. The system of claim 1, wherein the TR-DCS source comprises a seed light source and an amplifier.

12. The system of claim 11, wherein the seed light source is a continuous wave seed light source and the amplifier is a pulsed amplifier.

13. The system of claim 11, wherein the seed light source is a pulsed seed light source and the amplifier is a continuous wave amplifier.

14. The system of claim 11, wherein the seed light source is a pulsed seed light source and the amplifier is a pulsed amplifier.

15. The system of claim 14, wherein the pulse length of the pulses of light is determined by varying a pulse timing between the pulsed seed light source and the pulsed amplifier.

16. The system of claim 1, the system further comprising a second light source.

17. The system of claim 1, the system further comprising a second detector.

18. The system of claim 1, the system further comprising a trigger source configured to generate a trigger signal, the TR-DCS source configured to time its transmission relative to the trigger signal or the trigger source configured to time the trigger signal relative to the transmission from the TR-DCS source.

19. The system of claim 18, wherein the computer uses the trigger signal to determine a time of flight of received pulses of light.

20. The system of claim 1, wherein the TR-DCS detector is selected from the group consisting of a single-photon avalanche photodiode detector, a photomultiplier tube, a Si, Ge, InGaAs, PbS, PbSe or HgCdTe photodiode or PIN photodiode, phototransistors, MSM photodetectors, CCD and CMOS detector arrays, silicon photomultipliers, multi-pixel-photon-counters, and combinations thereof.

21. The system of claim 1, wherein the TR-DCS detector signal is an analog signal, a digital signal, a photon-counting signal, or a combination thereof.

22. The system of claim 1, the system further comprising one or more waveguides configured to couple the TR-DCS source to a target medium or configured to couple the target medium to the TR-DCS detector.

23. The system of claim 1, the system further comprising a time-resolved processor configured to process time-resolved aspects of the TR-DCS detector signal.

24. The system of claim 1, the system further comprising a signal processor configured to process correlation aspects of the TR-DCS detector signal.

25. The system of claim 1, wherein the system is contained in one or more handheld units.

26. A time-resolved diffuse correlation spectroscopy (TR-DCS) source comprising:
a light source configured to transmit pulses of light having a pulse length of between 1 ps and 10 ns into a target medium; and
a trigger source configured to generate a trigger signal that triggers the light source to emit the pulses of light and/or is correlated to the emission of the pulses of light from the light source,
the light source further configured to transmit the pulses of light into the target medium with either an average power of between 10 μW and 10 W or a coherence length of between 0.01 mm and a transform limit of the pulses of light.

27. The TR-DCS source of claim 26, wherein the light source is configured to transmit the pulses of light into the target medium with an average power of between 10 μW and 10 W.

28. The TR-DCS source of claim 26, wherein the light source is configured to transmit the pulses of light into the target medium with a coherence length of between 0.01 mm and a transform limit of the pulses of light.

29. A method for making a time-resolved diffuse correlation spectroscopy (TR-DCS) measurement of scattering particle dynamics within a target medium, the method comprising:
a) coupling a TR-DCS source and a TR-DCS detector to the target medium, the TR-DCS source configured to emit pulses of light having a pulse length of between 1 ps and 10 ns;
b) transmitting a first pulse of light from the TR-DCS source into the target medium, the first pulse of light comprising a plurality of photons;
c) receiving at least a portion of the plurality of photons at the TR-DCS detector after passing through the target medium, thereby generating a TR-DCS detector signal including a timing information and a correlation information for the at least a portion of the plurality of photons;
d) determining, using a processor, the timing information, the correlation information, and one or more equations relating time of flight and correlation to dynamics, a dynamics of the target medium; and
e) generating a report including the dynamics of the target medium.

30. The method of claim 29, wherein the transmitting of step b) comprises electronically or optically pulsing the TR-DCS source.

31. The method of claim 29, wherein the transmitting of step b) includes amplifying a non-amplified source to generate the at least one of the pulses of light.

32. The method of claim 29, wherein the TR-DCS source includes a seed light source and an amplifier.

33. The method of claim 32, wherein the seed light source is a continuous wave seed light source and the amplifier is a pulsed amplifier, and transmitting of step b) comprises seeding the pulsed amplifier with continuous wave seed light from the continuous wave seed light source.

34. The method of claim 32, wherein the seed light source is a pulsed seed light source and the amplifier is a continuous wave amplifier.

35. The method of claim 32, wherein the seed light source is a pulsed seed light source and the amplifier is a pulsed amplifier.

36. The method of claim 35 wherein the pulse length of the first pulse of light transmitted in step b) is determined by varying a pulse timing between the pulsed seed light source and the pulsed amplifier.

37. The method of claim 29, wherein the TR-DCS detector signal thereby generated by the receiving of step c) is an analog signal, a digital signal, or a combination thereof.

38. The method of claim 37, wherein the TR-DCS detector signal thereby generated by the receiving of step c) is the analog signal.

39. The method of claim 37, wherein the TR-DCS detector signal thereby generated by the receiving of step c) is the digital signal.

40. The method of claim 29, wherein the TR-DCS detector is a gated detector and the receiving of step c) involves gated detection.

41. The method of claim 40, wherein the receiving of step c) involves deactivating the gated detector during an initial time period and activating the gated detector during a subsequent time period.

42. The method of claim 41, wherein the initial time period is selected to at least partially coincide with the transmitting the first pulse of light of step b).

43. The method of claim 29, 37, 38, 39, or 40, wherein the timing information includes a time of flight tag for each of the at least a portion of the plurality of photons and the correlation information includes an arrival tag for each of the at least a portion of the plurality of photons.

44. The method of claim 43, wherein the determining of step d) includes selecting a subset of the at least a portion of the plurality of photons based on the time of flight tag for the subset falling within a pre-determined range and determining based on the timing information and the correlation information for the subset.

45. The method of claim 44, wherein a maximum of the pre-determined range minus a minimum of the pre-determined range is equal to or less than 2 times a coherence length of the TR-DCS light source.

46. The method of claim 44, wherein the pre-determined range is between 1 ps and 100 ns.

47. The method of claim 44, wherein the determining of step d) includes selecting a second subset of the at least a portion of the plurality of photons based on the time of flight tag for the second subset falling within a second pre-determined range and determining based on the timing information and the correlation information for the second subset.

48. The method of claim 29, 37, 38, 39, or 40, wherein the determining of step d) includes determining at two or more different time windows, thereby providing depth-dependent information about the dynamics of the target medium.

49. The method of claim 29, 37, 38, 39, or 40, wherein the TR-DCS detector signal thereby generated by the receiving of step c) includes wavelength information, and the determining of step d) uses the wavelength information.

50. The method of claim 49, wherein the wavelength information is used to enhance depth discrimination.

51. The method of claim 29, wherein steps a), b), and c), are repeated with a different distance between the TR-DCS source and the TR-DCS detector.

52. The method of claim 51, wherein the determining of step d) uses the different distance.

53. The method of claim 52, wherein the determining of step d) compensates for differences in the timing information due to the different distance.

54. The method of claim 29, wherein step a) further includes coupling a second TR-DCS detector to the target medium, the second TR-DCS detector positioned at a different distance from the TR-DCS source than the TR-DCS detector, wherein step c) further includes receiving at least a second portion of the plurality of photons at the second TR-DCS detector, thereby generating a second TR-DCS detector signal including a second timing information and a second correlation information for the at least a second portion of the plurality of photons, and wherein the determining of step d) uses the second timing information and the second correlation information.

55. The method of claim 54, wherein the determining of step d) uses the different distance.

56. The method of claim 55, wherein the determining of step d) compensates for differences in the timing information and the second timing information due to the different distance.

57. The method of claim 29, wherein the first pulse of light has a wavelength of between 400 nm and 1500 nm.

58. The method of claim 29, the method further comprising:
coupling a second DCS source to the medium, the second DCS source configured to emit continuous wave light having a coherence length sufficient for taking DCS measurements;
transmitting the continuous wave light from the DCS source into the medium; and
acquiring, using the DCS detector, the continuous wave light after the continuous wave light has traveled through the medium.

59. The method of claim 29, the method further comprising:
coupling a second DCS detector to the medium; and
receiving, using the second DCS detector, at least a second portion of the plurality of photons after passing through the target medium.

60. The method of claim 29, wherein the determining of step d) is achieved using a path length dependent autocorrelation function.

61. The method of claim 29, wherein the determining of step d) includes fitting data.

62. The method of claim 61, wherein the fitting data is achieved using a slope of a plot of correlation decay rate versus path length.

63. The method of claim 29, the method further comprising:
a1) optionally coupling a second TR-DCS source and/or a second TR-DCS detector to the target medium, the second TR-DCS source configured to emit second pulses of light having a pulse length of between 1 ps and 10 ns;
b1) transmitting a second pulse of light from the TR-DCS source or the second TR-DCS source into the target medium, the second pulse of light comprising a second plurality of photons;
c1) receiving at least a portion of the second plurality of photons at the TR-DCS detector or the second TR-DCS detector after passing through the target medium, thereby generating a second TR-DCS detector signal including a second timing information and a second correlation information for the at least a portion of the second plurality of photons,
the determining of step d) using the second timing information and the second correlation information.

64. The method of claim 63, wherein the first pulse of light and the second pulse of light have different wavelengths.

65. The method of claim 64, wherein the determining of step d) includes determining one or more properties of at least two distinct species of the target medium.

66. The method of claim 65, wherein the one or more properties of the at least two distinct species of the target medium include a concentration of the at least two distinct species.

67. The method of claim 65, wherein the at least two distinct species include oxyhemoglobin and deoxyhemoglobin.

68. The method of claim 29, wherein the dynamics of the target medium include a fluid flow within the target medium.

69. The method of claim 68, wherein the target medium is tissue and the fluid flow within the target medium is a blood flow within the tissue.

70. The method of claim 29, the method further comprising:
prior to the receiving of step c), gating the TR-DCS detector such that the TR-DCS detector signal is generated for a gated subset of the at least a portion of the plurality of photons received at the TR-DCS detector within a pre-determined gating time window.

71. The method of claim 29, the method further comprising:
prior to the determining of step d), gating the TR-DCS detector signal to include the timing information and the correlation information for a gated subset of the at least a portion of the plurality of photons within a pre-determined gating time window and to exclude the timing information and the correlation information for the at least a portion of the plurality of photons outside the gated subset.

72. The method of claim 71, the method further comprising repeating the gating, the determining of step d), and the generating of step e) for a different gated subset of the at least a portion of the plurality of photons within a second pre-determined gating time window.

73. A method of making a time-resolved diffuse correlation spectroscopy (TR-DCS) measurement of a target medium, the method comprising:
a) coupling a TR-DCS source to the target medium;
b) emitting a first pulse of light from the TR-DCS source into the target medium, the first pulse of light having a first pulse length of between 1 ps and 10 ns, the first pulse of light comprising a plurality of photons;
c) multiplexing at least a portion of the plurality of photons after passing through the target medium with a reference pulse of light emitted from the TR-DCS source or a different light source, thereby generating a multiplexed optical signal, the reference pulse of light has not passed through the target medium, the reference pulse of light having a reference pulse length that is the same or different than the first pulse length, the reference pulse length is between 1 ps and 100 ns;
d) receiving the multiplexed optical signal at an optical detector, thereby generating a detector signal including timing information and correlation information for the at least a portion of the plurality of photons;
e) determining, using a processor, the timing information, the correlation information, and one or more equations relating time of flight and correlation to dynamics, dynamics of the target medium; and
f) generating a report including the dynamics of the target medium.

74. A method of making a time-gated or time-tagged diffuse correlation spectroscopy (DCS) measurement of a target medium, the method comprising:
a) coupling a DCS source and a DCS detector to a surface of the target medium;
b) transmitting a plurality of photons from the DCS source into the target medium, each emitted photon emitted at a known emission time;
c) waiting a length of time for at least a portion of the plurality of photons to propagate through the medium from the DCS source to the DCS detector;
d) detecting the at least a portion of the plurality of photons using the DCS detector, each detected photon of the at least a portion of the plurality of photons detected at a known detection time;
e) determining a transit time for each of the at least a portion of the plurality of photons;
f) determining, using photons where the transit time that exceeds a pre-determined threshold, an inner dynamics of an inner portion of the target medium relative to the surface, or, using photons where the transit time is less than a pre-determined threshold, a superficial dynamics of a superficial layer of the target medium relative to the surface; and
g) generating a report including the inner dynamics or the superficial dynamics.

75. The method of claim 74, wherein the determining of step e) include subtracting the known emission time from the known detection time for each of the at least a portion of the plurality of photons.

76. The method of claim 74, wherein the transmitting of step b) comprises electronically or optically pulsing the DCS source.

77. The method of claim 74, wherein the transmitting of step b) includes amplifying a non-amplified source to generate the plurality of photons.

78. The method of claim 74, wherein the DCS source includes a seed light source and an amplifier.

79. The method of claim 78, wherein the seed light source is a continuous wave seed light source and the amplifier is a pulsed amplifier, and transmitting of step b) comprises seeding the pulsed amplifier with continuous wave seed light from the continuous wave seed light source.

80. The method of claim 78, wherein the seed light source is a pulsed seed light source and the amplifier is a continuous wave amplifier.

81. The method of claim 78, wherein the seed light source is a pulsed seed light source and the amplifier is a pulsed amplifier.

82. The method of claim 81 wherein a pulse length of the plurality of photons transmitted in step b) is determined by varying a pulse timing between the pulsed seed light source and the pulsed amplifier.

83. The method of claim 74, wherein the detecting of step d) thereby generates an analog signal, a digital signal, or a combination thereof.

84. The method of claim 83, wherein the detecting of step d) thereby generates the analog signal.

85. The method of claim 83, wherein the detecting of step d) thereby generates the digital signal.

86. The method of claim 74, wherein the DCS detector is a gated detector and the detecting of step d) involves gated detection.

87. The method of claim 86, wherein the detecting of step d) involves deactivating the gated detector during an initial time period and activating the gated detector during a subsequent time period.

88. The method of claim 87, wherein the initial time period is selected to at least partially coincide with the transmitting the first pulse of light of step b).

89. The method of claim 74, 83, 84, 85, or 86, wherein the TR-DCS detector signal thereby generated by the detecting of step d) includes wavelength information, and the determining of step f) uses the wavelength information.

90. The method of claim 89, wherein the wavelength information is used to enhance depth discrimination.

91. The method of claim 74, wherein steps a), b), c), and d) are repeated with a different distance between the DCS source and the DCS detector.

92. The method of claim 91, wherein the determining of step f) uses the different distance.

93. The method of claim 92, wherein the determining of step f) compensates for differences in the transit time due to the different distance.

94. The method of claim 74, wherein step a) further includes coupling a second DCS detector to the target medium, the second DCS detector positioned at a different distance from the DCS source than the DCS detector, wherein step d) further includes detecting at least a second portion of the plurality of photons using the second DCS detector, each detected photon of the at least a second portion of the plurality of photons detected at a second known detection time, and wherein the determining of step e) uses the second known detection time.

95. The method of claim 94, wherein the determining of step f) uses the different distance.

96. The method of claim 95, wherein the determining of step f) compensates for differences in the transit time and the second transit time due to the different distance.

97. The method of claim 74, wherein the plurality of photons has a wavelength of between 400 nm and 1500 nm.

98. The method of claim 74, the method further comprising:
coupling a second DCS source to the medium, the second DCS source configured to emit continuous wave light having a coherence length sufficient for taking DCS measurements;
transmitting the continuous wave light from the DCS source into the medium; and
acquiring, using the DCS detector, the continuous wave light after the continuous wave light has traveled through the medium.

99. The method of claim 74, the method further comprising:
coupling a second DCS detector to the medium; and
receiving, using the second DCS detector, at least a second portion of the plurality of photons after passing through the target medium.

100. The method of claim 74, wherein the determining of step f) is achieved using a path length dependent autocorrelation function.

101. The method of claim 74, wherein the determining of step f) includes fitting data.

102. The method of claim 101, wherein the fitting data is achieved using a slope of a plot of correlation decay rate versus path length.

103. The method of claim 74, the method further comprising:
a1) optionally coupling a second DCS source and/or a second DCS detector to the target medium, the second DCS source configured to transmit a second plurality of photons into the target medium;
b1) transmitting a second plurality of photons from the DCS source or the second DCS source into the target medium;
c1) waiting a second length of time for at least a portion of the second plurality of photons to propagate through the medium from the DCS source or the second DCS source to the DCS detector or the second DCS detector;
d1) detecting the at least a portion of the second plurality of photons using the DCS detector or the second DCS detector, each detected photon of the at least a portion of the second plurality of photons detected at a known time;
e1) determining a second transit time for each of the at least a portion of the second plurality of photons,
the determining of step f) using the second transit time.

104. The method of claim 103, wherein the first plurality of photons and the second plurality of photons have different wavelengths.

105. The method of claim 104, wherein the determining of step f) includes determining one or more properties of at least two distinct species of the target medium.

106. The method of claim 105, wherein the one or more properties of the at least two distinct species of the target medium include a concentration of the at least two distinct species.

107. The method of claim 106, wherein the at least two distinct species include oxyhemoglobin and deoxyhemoglobin.

108. The method of claim 74, wherein the inner dynamics and/or the superficial dynamics of the target medium include a fluid flow within the target medium.

109. The method of claim 108, wherein the target medium is tissue and the fluid flow within the target medium is a blood flow within the tissue.
